(19) United States
(12) Patent Application Publication
(10) Pub. No.: US 2007/0092881 A1
(43) Pub. Date: Apr. 26, 2007

(54) Gene markers of tumor metastasis

(75) Inventors: Yasuyuki Ohnishi, Yokohama (JP); Masato Nakamura, Tokyo (JP); Hiroshi Suemizu, Isehara (JP); Makoto Monnai, Yokohama (JP)

Correspondence Address: HELLER EHRMAN LLP, 275 MIDDLEFIELD ROAD, MENLO PARK, CA, 94025-3506, US

(73) Assignees: CENTRAL INSTITUTE FOR EXPERIMENTAL ANIMALS, Kawasaki-shi (JP); CHUGAI PHARMACEUTICAL, INC., Tokyo (JP); CENTER FOR THE ADVANCEMENT OF HEALTH AND BIOSCIENCE, USA, Menlo Park, CA (US)

(21) Appl. No.: 10/954,495

(22) Filed: Sep. 29, 2004

Related U.S. Application Data
(63) Continuation-in-part of application No. 10/871,186, filed on Jun. 18, 2004.
(60) Provisional application No. 60/487,044, filed on Jul. 10, 2003.

(51) Int. Cl. A61K 49/00; C12Q 1/68

(52) U.S. Cl. 435/6; 800/3

(57) ABSTRACT

The invention concerns the study of tumor metastasis in a NOD/SCID/γ_{c}^{null} transgenic mouse model and the identification of tumor markers, including markers of tumors with high metastatic potential, using information obtained in this mouse model.

This application is a continuation-in-part of copending U.S. application Ser. No. 10/871,186 filed on Jun. 18, 2004, which claims the benefit of the priority under 35 U.S.C. § 119(e) of provisional application Ser. No. 60/487,044 filed on Jul. 10, 2003.

### BACKGROUND OF THE INVENTION

1. Field of the Invention

The present invention relates to the identification of genes associated with tumor metastasis, using a transgenic mouse model. In particular, the invention concerns establishment and testing of human cancer cell lines with high metastatic potential in NOD/SCID/γcnull (NOG) mice, and the identification of genes selectively expressed in strongly metastatic cancer cells.

2. Related Art

Immunodeficient mice, such as athymic nude mice, C.B-17/severe combined immunodeficiency (scid) mice and NOD/SCID mice have been widely used as animal models in cancer metastasis research (Bruns et al., Int. J. Cancer 10:102(2):101-8 (2002); Ohta et al., Jpn. J. Cancer Chemother. 23:1669-72 (1996); Jimenez et al., Ann. Surg. 231:644-54 (2000)). Thus, such mouse models have been used for preclinical testing of new cancer drugs and for the detection of metastasis related genes (Bruns et al., supra; Ohta et al., supra; Jimenez et al. supra; Hotz et al., Pancreas 26:E89-98 (2003); Tarbe et al., Anticancer Res. 21:3221-8 (2001)). However, the use of these models for studying the metastases of human cancer cells has so far been limited, primarily due to the low efficiency of the incidence of cancer metastasis in the recipient mice, and the large cell number required to achieve the desired results.

Recently, to establish a more efficient animal recipient for xenotransplantation, a novel immunodeficiency mouse, NOD/SCID/γ_{c}^{null} (also referred to as NOD/ShiJic-scid with γ_{c}^{null}, or NOG) has been developed. NOG transgenic mice have been described as an excellent recipient mouse model for engraftment of human cells (Ito et al., Blood 100:3175-82 (2002)), and for the study of the in vivo development of human T cells from CD34(+) cells (Saito et al., Int. Immunol. 14:1113-24 (2002)). When human cord blood stem cells (CBSC) were preserved in NOG mice, CBSC were differentiated to T lymphocytes and migrated to the peripheral lymphoid organs (Yahata et al., J. Immunol. 169:204-9 (2002)).

Metastasis, including hepatic metastasis, is often observed in human cancer, including pancreatic cancer even in early stage, cancers of the digestive tract, including colorectal cancer and gastrointestinal cancer, lung cancer, and the like, and is one of the most frequent causes of cancer deaths. New strategies are necessary to manage cancer metastases, which, in turn, require the availability of appropriate gene markers of metastasis, including, but not limited to, metastasis in the liver.

### SUMMARY OF THE INVENTION

The present invention concerns identification of genes that are selectively expressed in tumor cells with high tendency to metastasize relative to tumor cells with low metastatic potential.

In one aspect, the invention concerns tumor markers, in particular, gene markers of metastatic cancer, such as metastatic pancreatic cancer.

In one embodiment, gene markers are selected from the group consisting of TIS1 1B protein; prostate differentiation factor (PDF); glycoproteins hormone α-subunit; thrombopoietin (THPO); manic fringe homology (MFNG); complement component 5 (C5); jagged homolog 1 (JAG1); interleukin enhancer-binding factor (ILF); PCAF-associated factor 65 alpha; interleukin-12 α-subunit (IL-12-α); nuclear respiratory factor 0.1 (NRF1); stem cell factor (SCF); transcription factor repressor protein (PRD1-BF1); small inducible cytokine subfamily A member 1 (SCYA1). transducin β2 subunit; X-ray repair complementing defective repair in Chinese hamster cells 1; putative renal organic anion transporter 1; G1/S-specific cyclin E (CCNE); retinoic acid receptor-γ (RARG); S-100 calcium-binding protein A1; neutral amino acid transporter A (SATT); dopachrome tautomerase; ets transcription factor (NERF2); calcium-activated potassium channel β-subunit; CD27BP; keratin 10; 6-O-methylguanine-DNA-methyltransferase (MGMT); xeroderma pigmentosum group A complementing protein (XPA); CDC6-related protein; cell division protein kinase 4; nociceptin receptor; cytochrome P450 XXVIIB1; N-myc proto-oncogene; solute carrier family member 1 (SLC2A1); membrane-associated kinase myt1; casper, a FADD- and caspase-related inducer of apoptosis; and C-src proto-oncogene.

In another embodiment, the gene markers are selected from the group consisting of TIS1 1B protein; prostate differentiation factor (PDF); glycoproteins hormone α-subunit; thrombopoietin (THPO); manic fringe homology (MFNG); complement component 5 (C5); jagged homolog 1 (JAG1); interleukin enhancer-binding factor (ILF); PCAF-associated factor 65 alpha; interleukin-12 α-subunit (IL-12-o); nuclear respiratory factor 1 (NRF1); stem cell factor (SCF); transcription factor repressor protein (PRD1-BF1); and small inducible cytokine subfamily A member 1 (SCYA1).

In a further embodiment, the gene markers are selected from the group consisting of transducin β2 subunit; X-ray repair complementing defective repair in Chinese hamster cells 1; putative renal organic anion transporter 1; G1/S-specific cyclin E (CCNE); retinoic acid receptor-γ (RARG); S-100 calcium-binding protein A1; neutral amino acid transporter A (SATT); dopachrome tautomerase; ets transcription factor (NERF2); calcium-activated potassium channel β-subunit; CD27BP; keratin 10; 6-O-methylguanine-DNA-methyltransferase (MGMT); xeroderma pigmentosum group A complementing protein (XPA); CDC6-related protein; cell division protein kinase 4; nociceptin receptor; cytochrome P450 XXVIIB1; N-myc proto-oncogene; solute carrier family member 1 (SLC2A1); membrane-associated kinase myt1; casper, a FADD- and caspase-related inducer of apoptosis; and C-src proto-oncogene.

In a still further embodiment, the gene markers are selected from the group consisting of interleukin 1 receptor-like 1, parathyroid hormone-like hormone, parathyroid hormone-like peptide, regulator of G-protein signaling 4, gap junction protein beta 6, neuregulin 1 isoform SMDF, fungal sterol-C5-desaturase homolog, G protein-coupled receptor, METH1 protein (ADAMTS1), METH1 protein (near ADAMTS15), BH-protocadherin (brain-heart), upregulated by 1,25-dihydroxyvitamin D-3, lipocalin 2 (oncogene 24p3) (LCN2), argininosuccinate synthetase (ASS), extrecellular matrix protein 1 (ECM1), S100 calcium-binding protein A4 (S100Z4), solute carrier family 6 (neurotransmitter transporter) mem1, serine (or cysteine) proteinase inhibitor, clade B (ovalbumin), tissue plasminogen activator (PLAT), EST DKFZp666M1410, C100 calcium-binding protein A8 (calgranulin A) (S100A8), EST (MGC:10500), placenta-specific 8 (PLAC8), interferon-inducible guanylate binding protein 2, matrix metalloproteinase 7, mucin 1 (transmembrane), EST nasopharyngal carcinoma-associated antigen/LOC5, megakaryocyte potentiating factor precursor, arrestin domain containing 4, interferon-gamma-inducible indoleamine 2,3-dioxygenase, DKFZP434G031 (Keratin 23), B-factor (properdin, complement), chloride intracellular channel 3, cystatin SN, carcinoembryonic antigen-related cell adhesion molecule 7, KIAA1358 (mucin 20), hypothetical protein (mucin 16=CA125), ring finger protein, aldehyde dehydrogenase 3 family member B1, DKFZp564P1263, serum amyloid A2, KiSS-1 metastasis-suppressor (KISS1), serum amyloid A2-alpha, protein kinase C-like 1, glucosaminyl (N-acetyl) transferase 3, mucintype, integrin-like protein beta 2 (antigen CD18, p95), kallikrein 8, NG22 protein, KLAA1359 (mucin 20), SCA2b (squamous cell carcinoma antigen SCCA, SERPINB4), carcinoembryonic antigen 2b, sphingosine-1-phosphate phosphatase 2, Susi domain containing 2, epithelial protein up-regulated in carcinoma, KIF21B kinesin family member 21B, carcinoembryonic antigen, polymeric immunoglobulin receptor/hepatocelullar carcinoma, gamma-aminobutyric acid (GABA) A receptor p1, synaptogyrin 3, NM_{—}024 783.1, alpha-1-antichymotrypsin precursor, and prostate stem cell antigen.

In a different aspect, the invention concerns an array comprising at least one of the genes listed above, or their expression products, immobilized on a solid support. The array can contain one or more of the listed genes, or their expression products, in any combination.

In various embodiments, the array displays at least 2, or at least 5, or at least 10, or at least 15, or at least 20, or at least 25, or at least 30, or at least 35, or at least 40, or at least 45, or at least 50, or at least 55, or at least 60, etc. of the listed genes, or their expression products.

In another embodiment, all genes that are overexpressed in tumor metastasis, or their expression products, are displayed.

In still further embodiments, all genes that are under-expressed in tumor metastasis, or their expression product, are displayed.

In a different aspect, the invention concerns a method for predicting the likelihood of tumor metastasis in a patient, comprising determining the expression level of the RNA transcript of one or more genes selected from the group consisting of TIS1 1B protein; prostate differentiation factor (PDF); glycoproteins hormone α-subunit; thrombopoietin (THPO); manic fringe homology (MFNG); complement component 5 (C5); jagged homolog 1 (JAG1); interleukin enhancer-binding factor (ILF); PCAF-associated factor 65 alpha; interleukin-12 α-subunit (IL-12-α); nuclear respiratory factor 1 (NRF1); stem cell factor (SCF); transcription factor repressor protein (PRD1-BF1); small inducible cytokine subfamily A member 1 (SCYA1), transducin P2 subunit; X-ray repair complementing defective repair in Chinese hamster cells 1; putative renal organic anion transporter 1; G1/S-specific cyclin E (CCNE); retinoic acid receptor-γ (RARG); S-100 calcium-binding protein A1; neutral amino acid transporter A (SATT); dopachrome tautomerase; ets transcription factor (NERF2); calcium-activated potassium channel β-subunit; CD27BP; keratin 10; 6-O-methylguanine-DNA-methyltransferase (MGMT); xeroderma pigmentosum group A complementing protein (XPA); CDC6-related protein; cell division protein kinase 4; nociceptin receptor; cytochrome P450 XXVIIB1; N-myc proto-oncogene; solute carrier family member 1 (SLC2A1); membrane-associated kinase myt1; casper, a FADD- and caspase-related inducer of apoptosis; and C-src proto-oncogene, or their expression products, in a test biological sample comprising cancer cells obtained from said patient, relative to a reference biological sample, wherein differential expression is indicative of an increased likelihood of tumor metastasis.

In yet another aspect, the invention concerns a method for predicting the likelihood of tumor metastasis in a patient, comprising determining the expression level of the RNA transcript of one or more genes selected from the group consisting of interleukin 1 receptor-like 1, parathyroid hormone-like hormone, parathyroid hormone-like peptide, regulator of G-protein signaling 4, gap junction protein beta 6, neuregulin 1 isoform SMDF, fungal sterol-C5-desaturase homolog, G protein-coupled receptor, METH1 protein (ADAMTS1), METH1 protein (near ADAMTS15), BH-protocadherin (brain-heart), upregulated by 1,25-dihydroxyvitamin D-3, lipocalin 2 (oncogene 24p3) (LCN2), argininosuccinate synthetase (ASS), extrecellular matrix protein 1 (ECM1), S100 calcium-binding protein A4 (S100Z4), solute carrier family 6 (neurotransmitter transporter) mem1, serine (or cysteine) proteinase inhibitor, clade B (ovalbumin), tissue plasminogen activator (PLAT), EST DKFZp666M1410, C100 calcium-binding protein A8 (calgranulin A) (S100A8), EST (MGC:10500), placenta-specific 8 (PLAC8), interferon-inducible guanylate binding protein 2, matrix metalloproteinase 7, mucin 1 (transmembrane), EST nasopharyngal carcinoma-associated antigen/LOC5, megakaryocyte potentiating factor precursor, arrestin domain containing 4, interferon-gamma-inducible indoleamine 2,3-dioxygenase, DKFZP434G031 (Keratin 23), B-factor (properdin, complement), chloride intracellular channel 3, cystatin SN, carcinoembryonic antigen-related cell adhesion molecule 7, KIAA1358 (mucin 20), hypothetical protein (mucin 16=CA125), ring finger protein, aldehyde dehydrogenase 3 family member B1, DKFZp564P1263, serum amyloid A2, KiSS-1 metastasis-suppressor (KISS1), serum amyloid A2-alpha, protein kinase C-like 1, glucosaminyl (N-acetyl) transferase 3, mucintype, integrin-like protein beta 2 (antigen CD18, p95), kallikrein 8, NG22 protein, KIAA1359 (mucin 20), SCA2b (squamous cell carcinoma antigen SCCA, SERPINB4), carcinoembryonic antigen 2b, sphingosine-1-phosphate phosphatase 2, Susi domain containing 2, epithelial protein up-regulated in carcinoma, KIF21B kinesin family member 21B, carcinoembryonic antigen, polymeric immunoglobulin receptor/hepatocelullar carcinoma, gamma-aminobutyric acid (GABA) A receptor p1, synaptogyrin 3, NM_{—}024 783.1, alpha-1-antichymotrypsin precursor, and prostate stem cell antigen, relative to a reference biological sample, wherein differential expression is indicative of an increased likelihood of tumor metastasis.

In a further embodiment, the invention concerns screening assay, comprising

(a) administering a candidate molecule to a tumor cell expressing one or more gene selected from the group consisting of TIS1 1B protein; prostate differentiation factor (PDF); glycoproteins hormone α-subunit; thrombopoietin (THPO); manic fringe homology (MFNG); complement component 5 (C5); jagged homolog 1 (JAG1); interleukin enhancer-binding factor (ILF); PCAF-associated factor 65 alpha; interleukin-12 α-subunit (IL-12-α); nuclear respiratory factor 1 (NRF1); stem cell factor (SCF); transcription factor repressor protein (PRDI-BF1); small inducible cytokine subfamily A member 1 (SCYA1), transducin P2 subunit; X-ray repair complementing defective repair in Chinese hamster cells 1; putative renal organic anion transporter 1; GltS-specific cyclin E (CCNE); retinoic acid receptor-γ (RARG); S-100 calcium-binding protein A1; neutral amino acid transporter A (SATT); dopachrome tautomerase; ets transcription factor (NERF2); calcium-activated potassium channel β-subunit; CD27BP; keratin 10; 6-O-methylguanine-DNA-methyltransferase (MGMT); xeroderma pigmentosum group A complementing protein (XPA); CDC6-related protein; cell division protein kinase 4; nociceptin receptor; cytochrome P450 XXVIIB31; N-myc proto-oncogene; solute carrier family member 1 (SLC2A1); membrane-associated kinase myt1; casper, a FADD- and caspase-related inducer of apoptosis; and C-src proto-oncogene, and

(b) determining the effect of the candidate molecule on the expression level of said gene or genes,

wherein the candidate molecule is identified as an anticancer agent, if (i) it is capable of suppressing the expression of one or more gene selected from the group consisting of TIS1 1B protein; prostate differentiation factor (PDF); glycoproteins hormone α-subunit; thrombopoietin (THPO); manic fringe homology (MFNG); complement component 5 (C5); jagged homolog 1 (JAG1); interleukin enhancer-binding factor (ILF); PCAF-associated factor 65 alpha; interleukin-12 α-subunit (IL-12-α); nuclear respiratory factor 1 (NRF1); stem cell factor (SCF); transcription factor repressor protein (PRD1-BF1); and small inducible cytokine subfamily A member 1 (SCYA1), or if (ii) it is capable of increasing the expression of one or more genes selected from the group consisting of transducin β2 subunit; X-ray repair complementing defective repair in Chinese hamster cells 1; putative renal organic anion transporter 1; G1/S-specific cyclin E (CCNE); retinoic acid receptor-γ (RARG); S-100 calcium-binding protein A1; neutral amino acid transporter A (SATT); dopachrome tautomerase; ets transcription factor (NERF2); calcium-activated potassium channel O-subunit; CD27BP; keratin 10; 6-O-methylguanine-DNA-methyltransferase (MGMT); xeroderma pigmentosum group A complementing protein (XPA); CDC6-related protein; cell division protein kinase 4; nociceptin receptor; cytochrome P450 XXVIUBI; N-myc proto-oncogene; solute carrier family member 1 (SLC2A1); membrane-associated kinase myt1; casper, a FADD- and caspase-related inducer of apoptosis; and C-src proto-oncogene.

In a still further aspect, the invention concerns a screening assay, comprising

(a) administering a candidate molecule to a tumor cell expressing one or more gene selected from the group consisting of interleukin 1 receptor-like 1, parathyroid hormone-like hormone, parathyroid hormone-like peptide, regulator of G-protein signaling 4, gap junction protein beta 6, neuregulin 1 isoform SMDF, fungal sterol-C5-desaturase homolog, G protein-coupled receptor, METH1 protein (ADAMTS1), METH1 protein (near ADAMTS15), BH-protocadherin (brain-heart), upregulated by 1,25-dihydroxyvitamin D-3, lipocalin 2 (oncogene 24p3) (LCN2), argininosuccinate synthetase (ASS), extrecellular matrix protein 1 (ECM1), S100 calcium-binding protein A4 (S100Z4), solute carrier family 6 (neurotransmitter transporter) mem1, serine (or cysteine) proteinase inhibitor, clade B (ovalbumin), tissue plasminogen activator (PLAT), EST DKFZp666M1410, C100 calcium-binding protein A8 (calgranulin A) (S100A8), EST (MGC:10500), placenta-specific 8 (PLAC8), interferon-inducible guanylate binding protein 2, matrix metalloproteinase 7, mucin 1 (transmembrane), EST nasopharyngal carcinoma-associated antigen/LOC5, megakaryocyte potentiating factor precursor, arrestin domain containing 4, interferon-gamma-inducible indoleamine 2,3-dioxygenase, DKFZP434G031 (Keratin 23), B-factor (properdin, complement), chloride intracellular channel 3, cystatin SN, carcinoembryonic antigen-related cell adhesion molecule 7, KIAA1358 (mucin 20), hypothetical protein (mucin 16=CA125), ring finger protein, aldehyde dehydrogenase 3 family member B1, DKFZp564P1263, serum amyloid A2, KiSS-1 metastasis-suppressor (KISS1), serum amyloid A2-alpha, protein kinase C-like 1, glucosaminyl (N-acetyl) transferase 3, mucintype, integrin-like protein beta 2 (antigen CD18, p95), kallikrein 8, NG22 protein, KIAA1359 (mucin 20), SCA2b (squamous cell carcinoma antigen SCCA, SERPINB4), carcinoembryonic antigen 2b, sphingosine-1-phosphate phosphatase 2, Susi domain containing 2, epithelial protein up-regulated in carcinoma, KIF21B kinesin family member 21B, carcinoembryonic antigen, polymeric immunoglobulin receptor/hepatocelullar carcinoma, gamma-aminobutyric acid (GABA) A receptor p1, synaptogyrin 3, NM_{—}024 783.1, alpha-1-antichymotrypsin precursor, and prostate stem cell antigen, relative to a reference biological sample, wherein differential expression is indicative of an increased likelihood of tumor metastasis interleukin 1 receptor-like 1, parathyroid hormone-like hormone, parathyroid hormone-like peptide, regulator of G-protein signaling 4, gap junction protein beta 6, neuregulin 1 isoform SMDF, fungal sterol-C5-desaturase homolog, G protein-coupled receptor, METH1 protein (ADAMTS1), METH1 protein (near ADAMTS15), BH-protocadherin (brain-heart), upregulated by 1,25-dihydroxyvitamin D-3, lipocalin 2 (oncogene 24p3) (LCN2), argininosuccinate synthetase (ASS), extrecellular matrix protein 1 (ECM1), S100 calcium-binding protein A4 (S100Z4), solute carrier family 6 (neurotransmitter transporter) mem1, serine (or cysteine) proteinase inhibitor, clade B (ovalbumin), tissue plasminogen activator (PLAT), EST DKFZp666M1410, C100 calcium-binding protein A8 (calgranulin A) (S100A8), EST (MGC:10500), placenta-specific 8 (PLAC8), interferon-inducible guanylate binding protein 2, matrix metalloproteinase 7, mucin 1 (transmembrane), EST nasopharyngal carcinoma-associated antigen/LOC5, megakaryocyte potentiating factor precursor, arrestin domain containing 4, interferon-gamma-inducible indoleamine 2,3-dioxygenase, DKFZP434G031 (Keratin 23), B-factor (properdin, complement), chloride intracellular channel 3, cystatin SN, carcinoembryonic antigen-related cell adhesion molecule 7, KIAA1358 (mucin 20), hypothetical protein (mucin 16=CA125), ring finger protein, aldehyde dehydrogenase 3 family member B1, DKFZp564P1263, serum amyloid A2, KiSS-1 metastasis-suppressor (KISS1), serum amyloid A2-alpha, protein kinase C-like 1, glucosaminyl (N-acetyl) transferase 3, mucintype, integrin-like protein beta 2 (antigen CD18, p95), kallikrein 8, NG22 protein, KLAA1359 (mucin 20), SCA2b (squamous cell carcinoma antigen SCCA, SERPINB4), carcinoembryonic antigen 2b, sphingosine-1-phosphate phosphatase 2, Susi domain containing 2, epithelial protein up-regulated in carcinoma, KIF21B kinesin family member 21B, carcinoembryonic antigen, polymeric immunoglobulin receptor/hepatocelullar carcinoma, gamma-aminobutyric acid (GABA) A receptor p1, synaptogyrin 3, NM_{—}024 783.1, alpha-1-antichymotrypsin precursor, and prostate stem cell antigen, relative to a reference biological sample, wherein differential expression is indicative of an increased likelihood of tumor metastasis, and

(b) determining the effect of the candidate molecule on the expression level of said gene or genes,

wherein the candidate molecule is identified as an anticancer agent, if (i) it is capable of suppressing the expression of one or more gene selected from the group consisting of upregulated by 1,25-dihydroxyvitamin D-3, lipocalin 2 (oncogene 24p3) (LCN2), argininosuccinate synthetase (ASS), extrecellular matrix protein 1 (ECM1), S100 calcium-binding protein A4 (S100Z4), solute carrier family 6 (neurotransmitter transporter) mem1, serine (or cysteine) proteinase inhibitor, clade B (ovalbumin), tissue plasminogen activator (PLAT), EST DKFZp666M1410, C100 calcium-binding protein A8 (calgranulin A) (S100A8), EST (MGC:10500), placenta-specific 8 (PLAC8), interferon-inducible guanylate binding protein 2, matrix metalloproteinase 7, mucin 1 (transmembrane), EST nasopharyngal carcinoma-associated antigen/LOC5, megakaryocyte potentiating factor precursor, arrestin domain containing 4, interferon-gamma-inducible indoleamine 2,3-dioxygenase, DKFZP434G031 (Keratin 23), B-factor (properdin, complement), chloride intracellular channel 3, cystatin SN, carcinoembryonic antigen-related cell adhesion molecule 7, KIAA1358 (mucin 20), hypothetical protein (mucin 16=CA125), ring finger protein, aldehyde dehydrogenase 3 family member B1, DKFZp564P1263, serum amyloid A2, KiSS-1 metastasis-suppressor (KISS1), serum amyloid A2-alpha, protein kinase C-like 1, glucosaminyl (N-acetyl) transferase 3, mucintype, integrin-like protein beta 2 (antigen CD18, p95), kallikrein 8, NG22 protein, KIAA1359 (mucin 20), SCA2b (squamous cell carcinoma antigen SCCA, SERPINB4), carcinoembryonic antigen 2b, sphingosine-1-phosphate phosphatase 2, Susi domain containing 2, epithelial protein up-regulated in carcinoma, KIF21B kinesin family member 21B, carcinoembryonic antigen, polymeric immunoglobulin receptor/hepatocelullar carcinoma, gamma-aminobutyric acid (GABA) A receptor p1, synaptogyrin 3, NM_{—}024 783.1, alpha-1-antichymotrypsin precursor, and prostate stem cell antigen, relative to a reference biological sample, wherein differential expression is indicative of an increased likelihood of tumor metastasis interleukin 1 receptor-like 1, parathyroid hormone-like hormone, parathyroid hormone-like peptide, regulator of G-protein signaling 4, gap junction protein beta 6, neuregulin 1 isoform SMDF, fungal sterol-C5-desaturase homolog, G protein-coupled receptor, METH1 protein (ADAMTS1), METH1 protein (near ADAMTS15), BH-protocadherin (brain-heart), upregulated by 1,25-dihydroxyvitamin D-3, lipocalin 2 (oncogene 24p3) (LCN2), argininosuccinate synthetase (ASS), extrecellular matrix protein 1 (ECM1), S100 calcium-binding protein A4 (S100Z4), solute carrier family 6 (neurotransmitter transporter) mem1, serine (or cysteine) proteinase inhibitor, clade B (ovalbumin), tissue plasminogen activator (PLAT), EST DKFZp666MI410, C100 calcium-binding protein A8 (calgranulin A) (S100A8), EST (MGC:10500), placenta-specific 8 (PLAC8), interferon-inducible guanylate binding protein 2, matrix metalloproteinase 7, mucin 1 (transmembrane), EST nasopharyngal carcinoma-associated antigen/LOC5, megakaryocyte potentiating factor precursor, arrestin domain containing 4, interferon-gamma-inducible indoleamine 2,3-dioxygenase, DKFZP434G031 (Keratin 23), B-factor (properdin, complement), chloride infracellular channel 3, cystatin SN, carcinoembryonic antigen-related cell adhesion molecule 7, KIAA1358 (mucin 20), hypothetical protein (mucin 16=CA125), ring finger protein, aldehyde dehydrogenase 3 family member B1, DKFZp564P1263, serum amyloid A2, KiSS-1 metastasis-suppressor (KISS1), serum amyloid A2-alpha, protein kinase C-like 1, glucosaminyl (N-acetyl) transferase 3, mucintype, integrin-like protein beta 2 (antigen CD18, p95), kallikrein 8, NG22 protein, KIAA1359 (mucin 20), SCA2b (squamous cell carcinoma antigen SCCA, SERPINB4), carcinoembryonic antigen 2b, sphingosine-1-phosphate phosphatase 2, Susi domain containing 2, epithelial protein up-regulated in carcinoma, KIF21B kinesin family member 21B, carcinoembryonic antigen, polymeric immunoglobulin receptor/hepatocelullar carcinoma, gamma-aminobutyric acid (GABA) A receptor p1, synaptogyrin 3, NM_{—}024 783.1, alpha-1-antichymotrypsin precursor, and prostate stem cell antigen, or if (ii) it is capable of increasing the expression of one or more genes selected from the group consisting of interleukin 1 receptor-like 1, parathyroid hormone-like hormone, parathyroid hormone-like peptide, regulator of G-protein signaling 4, gap junction protein beta 6, neuregulin 1 isoform SMDF, fungal sterol-C5-desaturase homolog, G protein-coupled receptor, METH1 protein (ADAMTS1), METH1 protein (near ADAMTS15), and BH-protocadherin (brain-heart).

In yet another aspect, the invention concerns a screening assay, comprising

determining the ability of a candidate molecule to modulate the expression of one or more gene selected from the group consisting of TIS1 1B protein; prostate differentiation factor (PDF); glycoproteins hormone α-subunit; thrombopoietin (THPO); manic fringe homology (MFNG); complement component 5 (C5); jagged homolog 1 (JAG1); interleukin enhancer-binding factor (ILF); PCAF-associated factor 65 alpha; interleukin-12 α-subunit (IL-12-α); nuclear respiratory factor 1 (NRF1); stem cell factor (SCF); transcription factor repressor protein (PRD1-BF1); small inducible cytokine subfamily A member 1 (SCYA1), transducin β2 subunit; X-ray repair complementing defective repair in Chinese hamster cells 1; putative renal organic anion transporter 1; G1/S-specific cyclin E (CCNE); retinoic acid receptor-γ (RARG); S-100 calcium-binding protein A1; neutral amino acid transporter A (SATT); dopachrome tautomerase; ets transcription factor (NERF2); calcium-activated potassium channel β-subunit; CD27BP; keratin 10; 6-O-methylguanine-DNA-methyltransferase (MGMT); xeroderma pigmentosum group A complementing protein (XPA); CDC6-related protein; cell division protein kinase 4; nociceptin receptor; cytochrome P450 XXVIIB1; N-myc proto-oncogene; solute carrier family member 1 (SLC2A1); membrane-associated kinase myt1; casper, a FADD- and caspase-related inducer of apoptosis; and C-src proto-oncogene, and identifying said candidate molecule as an anticancer agent when such modulation occurs.

The invention further concerns a screening assay, comprising determining the ability of a candidate molecule to modulate the expression of one or more gene selected from the group consisting of interleukin 1 receptor-like 1, parathyroid hormone-like hormone, parathyroid hormone-like peptide, regulator of G-protein signaling 4, gap junction protein beta 6, neuregulin 1 isoform SMDF, fungal sterol-C5-desaturase homolog, G protein-coupled receptor, METH1 protein (ADAMTS1), METH1 protein (near ADAMTS15), BH-protocadherin (brain-heart), upregulated by 1,25-dihydroxyvitamin D-3, lipocalin 2 (oncogene 24p3) (LCN2), argininosuccinate synthetase (ASS), extrecellular matrix protein 1 (ECM1), S100 calcium-binding protein A4 (S100Z4), solute carrier family 6 (neurotransmitter transporter) mem1, serine (or cysteine) proteinase inhibitor, clade B (ovalbumin), tissue plasminogen activator (PLAT), EST DKFZp666M1410, C100 calcium-binding protein A8 (calgranulin A) (S100A8), EST (MGC:10500), placenta-specific 8 (PLAC8), interferon-inducible guanylate binding protein 2, matrix metalloproteinase 7, mucin 1 (transmembrane), EST nasopharyngal carcinoma-associated antigen/LOC5, megakaryocyte potentiating factor precursor, arrestin domain containing 4, interferon-gamma-inducible indoleamine 2,3-dioxygenase, DKFZP434G031 (Keratin 23), B-factor (properdin, complement), chloride intracellular channel 3, cystatin SN, carcinoembryonic antigen-related cell adhesion molecule 7, KLIA1358 (mucin 20), hypothetical protein (mucin 16=CA125), ring finger protein, aldehyde dehydrogenase 3 family member B1, DKFZp564P1263, serum amyloid A2, KiSS-1 metastasis-suppressor (KISS1), serum amyloid A2-alpha, protein kinase C-like 1, glucosaminyl (N-acetyl) transferase 3, mucintype, integrin-like protein beta 2 (antigen CD18, p95), kallikrein 8, NG22 protein, KIAA1359 (mucin 20), SCA2b (squamous cell carcinoma antigen SCCA, SERPINB4), carcinoembryonic antigen 2b, sphingosine-1-phosphate phosphatase 2, Susi domain containing 2, epithelial protein up-regulated in carcinoma, KIF21B kinesin family member 21B, carcinoembryonic antigen, polymeric immunoglobulin receptor/hepatocelullar carcinoma, gamma-aminobutyric acid (GABA) A receptor p1, synaptogyrin 3, NM_{—}024 783.1, alpha-1-antichymotrypsin precursor, and prostate stem cell antigen, and identifying said candidate molecule as an anticancer agent when such modulation occurs.

### BRIEF DESCRIPTION OF THE DRAWINGS

FIGS. 1A and B illustrate the incidences of hepatic metastasis and the number of liver foci in NOG mice following the inoculation of 1×10⁴, 1×10³ and 1×10² cells of the indicated pancreatic adenocarcinoma cells lines (MIAPaCa-2, AsPC-1, PANC-1, Capan-1, and BxPC-3.

FIG. 2. Establishment of a the highly metastatic cell line, BxPC-3LM1, derived from the low metastatic cell line, BxPC-3.

FIG. 3. PCR data confirming that BxPC-3LM1 is a cell line of human origin, using β-actin specific primers.

FIG. 4. Microscopic view of the cell lines BxPC-3 and BxPC-3LM1 in vitro.

FIG. 5. Overview of establishing and testing the cell line BxPC-3LM1.

FIG. 6 is a scatter graph of microarray analysis of the BxPC-3LM1 and BxPC-3 cell lines. Signal intensity of each spot of gene chip microarray (gene expression) was plotted. The genes in the blue bottom area was omitted from further analysis due to low intensity. The genes in the red are were subjected to further analysis.

FIG. 7. To confirm the results of gene expression analysis, some genes which showed significant difference in their expression levels in BxPC-3 and BxPC-3LM1 were subjected to amplification by RT-PCR. The Figure shows the results of the RT-PCR analysis after varying numbers of amplification cycles.

Table 1. Hepatic metastasis after intrasplenic injection of various human pancreatic adenocarcinoma cell lines.

Table 2. Genes differentially expressed in cell lines with high metastasis potential relative to cell lines with low metastatic potential.

Table 3. Metastatic potentials of BxPC-3 and BxPC-3LM1 in the liver of NOG mouse.

Table 4 Metastatic potentials of BxPC-3 and BxPC-3LM1 in the liver of NOD/SCID mouse.

Table 5 Comparison of characteristics of BxPC-3 and BxPC-3LM1 cell lines.

Table 6 List and signal log ratio of genes differentially expressed between the BxPC-3 and BxPC-3LM1 cell lines, selected by microarray analysis.

### DETAILED DESCRIPTION OF THE PREFERRED EMBODIMENT

A. Definitions

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

The term “tumor,” as used herein, refers to all neoplastic cell growth and proliferation, whether malignant or benign, and all pre-cancerous and cancerous cells and tissues.

The terms “cancer” and “cancerous” refer to or describe the physiological condition in mammals that is typically characterized by unregulated cell growth. Examples of cancer include but are not limited to, carcinoma (epithelial), such as, pancreatic cancer, prostate cancer, breast cancer, colorectal cancer, gastrointestinal cancer, colon cancer, lung cancer, hepatocellular cancer, cervical cancer, ovarian cancer, liver cancer, bladder cancer, cancer of the urinary tract, thyroid cancer, renal cancer, melanoma, and brain cancer; and sarcoma (non-epithelial), such as, liposarcomas, leiomyosarcomas, rhabdomyosarcoma, synovial sarcoma, angiosarcoma, fibrosarcoma, malignant peripheral nerve tumor, gastrointestinal stromal tumor, desmoid tumor, Ewing's sarcoma, osteosarcoma, chondrosarcoma, leukemia, lymphoma and myeloma.

The term “metastasis” is used herein in the broadest sense and refers to the spread of tumor, e.g. cancer from one part of the body to another. Tumors formed from cells that have spread are called secondary tumors, and contain the same type of cells as the original (primary) tumor. Thus prostate cancer that has metastasized to liver or bone is not liver or bone cancer, rather metastasized prostate cancer, as it still contains prostate cancer cells, regardless of their location.

The “pathology” of cancer includes all phenomena that compromise the well-being of the patient. This includes, without limitation, abnormal or uncontrollable cell growth, metastasis, interference with the normal functioning of neighboring cells, release of cytokines or other secretory products at abnormal levels, suppression or aggravation of inflammatory or immunological response, neoplasia, premalignancy, malignancy, invasion of surrounding or distant tissues or organs, such as lymph nodes, etc.

The terms “differentially expressed gene,” “differential gene expression” and their synonyms, which are used interchangeably, refer to a gene whose expression is at a higher or lower level in one cell or cell type relative to another, or one patient or test subject relative to another. Thus, for example, differential gene expression can occur in normal cell/tissue/patient relative to a corresponding diseased cell/tissue/patient, or can reflect differences is gene expression pattern between different cell types or cells in different stages of development. The terms also include genes whose expression is activated to a higher or lower level at different stages of the same disease. It is also understood that a differentially expressed gene may be either activated or inhibited at the nucleic acid level or protein level, or may be subject to alternative splicing to result in a different polypeptide product. Such differences may, for example, be evidenced by a change in mRNA levels, surface expression, or secretion or other partitioning of a polypeptide. Differential gene expression may include a comparison of expression between two or more genes or their gene products, or a comparison of the ratios of the expression between two or more genes or their gene products, or a comparison of two differently processed products of the same gene. For the purpose of the present invention, “differential gene expression” is considered to be present when there is at least an about 2-fold, preferably at least about 2.5-fold, more preferably at least about 4-fold, even more preferably at least about 6-fold, most preferably at least about 10-fold difference between the expression of a given gene or gene product between the samples compared. The “reference biological sample” can, for example, be a normal sample, or a sample of a tumor of the same type, having low metastatic potential.

The term “microarray” refers to an ordered arrangement of hybridizable array elements on a substrate. The term specifically includes polynucleotide microarrays, such as cDNA and oligonucleotide microarrays, and protein arrays. In a particular embodiment, a microarray is an array of thousands of individual gene (DNA) sequences immobilized in a known order on a solid support. RNAs from different tissues are hybridized to the DNA on the chips. An RNA molecule will only bind to the DNA from which it was expressed. As a result, the relative expression of thousands of genes in biological samples (e.g. normal and diseased tissue, tissue treated or untreated with a certain drug, etc.) can be compared in a single assay. In a similar protein sequences can be displayed on a microarray chip and used to study protein-protein interactions, or differences in protein levels in different biological samples, e.g. tissues.

The term “polynucleotide,” generally refers to any polyribonucleotide or polydeoxribonucleotide, which may be unmodified RNA or DNA or modified RNA or DNA. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein includes triple-stranded regions comprising RNA or DNA or both RNA and DNA. The strands in such regions may be from the same molecule or from different molecules. The term includes DNAs (including cDNAs) and RNAs that contain one or more modified bases. Thus, DNAs or RNAs with backbones modified for stability or for other reasons are “polynucleotides” as that term is intended herein. Moreover, DNAs or RNAs comprising unusual bases, such as inosine, or modified bases, such as tritiated bases, are included within the term “polynucleotides” as defined herein. In general, the term “polynucleotide” embraces all chemically, enzymatically and/or metabolically modified forms of unmodified polynucleotides, as well as the chemical forms of DNA and RNA characteristic of viruses and cells, including simple and complex cells.

The term “oligonucleotide” refers to a relatively short polynucleotide, including, without limitation, single-stranded deoxyribonucleotides, single- or double-stranded ribonucleotides, RNA:DNA hybrids and double-stranded DNAs.

The terms “transgenic animal” and “transgenic mouse” as well we their grammatical equivalents, are used to refer to animals/mice deliberately produced to carry a gene from another animal.

The term “xenotransplantation” is used in the broadest sense and refers to the transfer of living cells, tissues or organs from one animal species into another, including humans.

B. Detailed Description

The practice of the present invention will employ, unless otherwise indicated, conventional techniques of molecular biology (including recombinant techniques), microbiology, cell biology, and biochemistry, which are within the skill of the art. Such techniques are explained fully in the literature, such as, “Molecular Cloning: A Laboratory Manual”, 2^{nd} edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology”, 4^{th} edition (D. M. Weir & C. C. Blackwell, eds., Blackwell Science Inc., 1987); “Gene Transfer Vectors for Mammalian Cells” (J. M. Miller & M. P. Calos, eds., 1987); “Current Protocols in Molecular Biology” (F. M. Ausubel et al., eds., 1987); “Transgenic Mouse: Methods and Protocols” (Methods in Molecular Biology, Clifton N. J., Vol. 209, M. H. Hofker et al., eds.).

The present invention provides a sensitive and reliable transgenic animal model for the study of tumor metastasis. In particular, the present invention provides a reproducible mouse model of hepatic metastasis, which involves the introduction of mammalian (e.g. human) cancer cells into NOG mice.

NOG mice were developed at the Central Institute for Experimental Animals (CIEA, Kawasaki, Japan), and are also described in co-pending U.S. application Ser. No. 10/221,549 filed on Oct. 25, 2001, and in PCT Publication No. WO 03/0182671, the entire disclosures of which are hereby expressly incorporated by reference.

In brief, to establish an improved animal recipient for xenotransplantation, NOD/SCID/γ_{c}^{null} (NOG) mice double homozygous for the severe combined immunodeficiency (SCID) mutation and interleukin-2Rγ (IL-2Rγ) allelic mutation (γ_{c}^{null}) were generated by 8 backcross matings of C57BL/6J-γ_{c}^{null} mice and NOD/Shi-scid mice. When human CD34+ cells from umbilical cord blood were transplanted into this strain, the engraftment rate in the peripheral circulation, spleen, and bone marrow were significantly higher than that in NOD/Shi-scid mice treated with anti-asialo GM1 antibody or in the P2-microglobulin-deficient. NOD/LtSz-scid (NOD/SCID/β₂ₘ^{null}) mice, which were as completely defective in NK cell activity as NOD/SCID/γ_{c}^{null} mice. The same high engraftment rate of human mature cells was observed in ascites when peripheral blood mononuclear cells were intraperitoneally transferred. In addition to the high engraftment rate, multilineage cell differentiation was also observed. Further, even 1×10(2) CD34+cells could grow and differentiate in this strain. Based on these results, the NOD/SCID/γ_{c}^{null} mice were described to be superior animal recipients for xenotransplantation, especially for human stem cell assays. For further details see, e.g. Hiramatsu et al., Blood 100:3175-82 (2002).

It has now been found that the NOG mice are a superior mouse model for the study of human cancer metastasis. As such, this model can be used, for example, to screen and evaluate anti-cancer drugs and anti-metastasis drug candidates, and for the detection/screening of genes related to cancer metastasis, which, in turn, find utility in the diagnosis and/or treatment of metastatic cancer, and related conditions, including gene therapy treatment of metastatic cancer.

The mouse model of the present invention is suitable for modeling and studying any kind of metastasis, including hepatic, bone, brain, and lung metastasis. Metastasis occurs in all types of cancers, including, without limitation, pancreatic cancer, prostate cancer, breast cancer, colorectal cancer, gastrointestinal cancer, colon cancer, lung cancer, hepatocellular cancer, cervical cancer, ovarian cancer, liver cancer, bladder cancer, cancer of the urinary tract, thyroid cancer, renal cancer, carcinoma, melanoma, and brain cancer. Although the invention will be illustrated by analyzing hepatic metastasis of human pancreatic cancer, it is not so limited. The NOG mouse model can also be used to study metastases originating from other types of cancer at any location, including liver, bone, brain and liver.

Methods of xenotransplantation are well known in the art, and described, for example, in the following references, which are incorporated herein in their entirety: Fiebig et al., “Human Tumor Xenografts: Predictivity, Characterization and Discovery of New Anticancer Agents,” in Contributions to Oncology: Relevance of Tumor Models for Anticancer Drug Development, Fiebig & Burger, eds. (Basel, Karger 1999), vol. 54, pp. 29-50; Berger et al., “Establishment and Characterization of Human Tumor Xenografts in Thymus-Aplastic Nude Mice,” in Immunodeficient Mice in Oncology, Fiebig & Berger, eds. (Basel, Karger 1992), pp. 23-46; Fiebig & Burger, “Human Tumor Xenografts and Explants,” in Models in Cancer Research, Teicher, ed. (Humana Press 2002) pp. 113-137.

A specific method of xenotransplantion is also described in Example 1 below.

In general, mammalian tumor specimens, preferably human tumor specimens, may be obtained and implanted into mice, preferably athymic nude mice. The tumor specimens may be obtained by any method known in the art. In one embodiment the tumor specimens are surgically resected, such as in a biopsy or in the process of surgery to remove the tumor from the mammal. In another embodiment the tumor specimen is obtained by purifying circulating tumor cells from the mammals blood.

Typically, cancer cells are transplanted into mice via tail vein injection, with or without prior immune-suppression, such as a sublethal dose of whole body irradiation and/or the administration of an immunosuppressant. For study of hepatic metastases, the cancer cells may be introduced into the animals by intrasplenic (portal vein) injection using an appropriate indwelling catheter. Pulmonary metastasis can be established, for example, by intravenous injection of tumor cells into the recipient animals, for example as described in Worth and Kleinerman; Clin Exp. Metastasis 17:501-6 (1999). The tumor cells may originate from tumor (cancer) cell lines, and from primary tumors (e.g. cancer) obtained from human or non-human subjects.

To study bone metastasis, macroscopic fragments of human fetal bone or mouse bone, may be implanted into NOG mice. A few weeks later, human tumor (cancer) cell lines or cells of primary tumors (cancer) can be injected either intravenously (colonization assay), or directly into the implanted tissue fragments. Tumor metastasis can be monitored by methods known in the art, including various imaging techniques and histologic examination.

Human xenografts are considered highly predictive of tumor behavior within the donor patient, as the xenograft grows as a solid tumor, differentiates, and develops a stroma, vasculature, and a central necrosis. In most cases, xenografts retain most of the molecular, histological, and pathophysiological characteristics of the fresh patient-derived tumor.

When used for drug screening, following the engraftment of xenogenic tumor cells (either from cell lines or from primary tumors), the NOG mice that have developed metastatic cancer can be treated with the test compound(s), and any change in the number, size or other properties of the metastatic nodules as a result of drug treatment, and the viability of the test animals are monitored relative to untreated and/or positive control, where the positive control typically is an animal treated with a know anti-metastatic compound. The administration of the test compounds can be performed by any suitable route, including, for example, oral, transdermal, intravenous, infusion, intramuscular, etc. administration. Results obtained in this model can then be validated by follow-up pharmacokinetic, toxicologic, biochemical and immunologic studies, and ultimately human clinical studies.

The NOG mouse model can also be used to study targeted gene delivery to metastatic nodules in vivo, for example by portal vein infusion of a retroviral vector. In particular, this NOG model can be used to study the feasibility of gene transfer to target tumor metastasis, to monitor the duration and level of gene expression and the degree of therapeutic effect, to optimize the dosing regimen and/or mode of administration, to study the dissemination of the gene transfer vector to non-targeted tissues (which provides information about potential toxicity), and the like.

Gene delivery most commonly is performed using retroviral vectors by techniques well known in the art. Retroviruses are enveloped viruses containing a single stranded RNA molecule as their genome. Following infection, the viral genome is reverse transcribed into double stranded DNA, which integrates into the host genome where it is expressed. The viral genome contains at least three genes: gag (coding for core proteins), pol (coding for reverse transcriptase) and env (coding for the viral envelope protein). At each end of the genome are long terminal repeats (LTRs) which include promoter/enhancer regions and sequences involved with viral integration. In addition there are sequences required for packaging the viral DNA and RNA splice sites in the env gene. Retroviral vectors used in mouse models are most frequently based upon the Moloney murine leukemia virus (Mo-MLV). In addition, lentiviruses can, for example, be used for gene transfer into experimental animals, such as NOG mice.

Gene delivery can also be performed by adenoviral vectors. Adenoviroses are non-enveloped, icosahedral viruses with linear double-stranded DNA genomes. Adenoviruses infect non-dividing cells by interacting with cell surface receptors, and enter cells by endocytosis. Since the genome of adenoviruses cannot integrate with the host cell genome, the expression from adenoviral vectors is transient.

As part of the present invention, NOG mice were used to study differential gene expression between cells exhibiting high tendency to metastasize and cells with low metastatic potential. In particular, gene expression patterns in highly metastatic cell lines and cell lines with low metastatic potential were compared, and differentially expressed genes were identified.

Methods of gene expression profiling are well known in the art and include methods based on hybridization analysis of polynucleotides, methods based on sequencing of polynucleotides, and proteomics-based methods. The most commonly used methods known in the art for the quantification of mRNA expression in a sample include northern blotting and in situ hybridization (Parker & Barnes, Methods in Molecular Biology 106:247-283 (1999)); RNAse protection assays (Hod, Biotechniques 13:852-854 (1992)); and PCR-based methods, such as reverse transcription polymerase chain reaction (RT-PCR) (Weis et al., Trends in Genetics 8:263-264 (1992)). Alternatively, antibodies may be employed that can recognize specific duplexes, including DNA duplexes, RNA duplexes, and DNA-RNA hybrid duplexes or DNA-protein duplexes. Representative methods for sequencing-based gene expression analysis include Serial Analysis of Gene Expression (SAGE), and gene expression analysis by massively parallel signature sequencing (MPSS).

One of the most sensitive and most flexible quantitative PCR-based gene expression profiling methods is RT-PCR, which can be used to compare mRNA levels in different sample populations, including various tumor cells or tissues, to characterize patterns of gene expression.

As RNA cannot serve as a template for PCR, the first step in gene expression profiling by RT-PCR is the reverse transcription of the RNA template into cDNA, followed by its exponential amplification in a PCR reaction. The two most commonly used reverse transcriptases are avilo myeloblastosis virus reverse transcriptase (AMV-RT) and Moloney murine leukemia virus reverse transcriptase (MMLV-RT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, Calif., USA), following the manufacturer's instructions. The derived cDNA can then be used as a template in the subsequent PCR reaction.

Although the PCR step can use a variety of thermostable DNA-dependent DNA polymerases, it typically employs the Taq DNA polymerase, which has a 5′-3′ nuclease activity but lacks a 3′-5′ proofreading endonuclease activity. Thus, TaqMan® PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

TaqMan® RT-PCR can be performed using commercially available equipment, such as, for example, ABI PRISM 7700™ Sequence Detection System™ (Perkin-Elmer-Applied Biosystems, Foster City, Calif., USA), or Lightcycler (Roche Molecular Biochemicals, Mannheim, Germany).

A more recent variation of the RT-PCR technique is the real time quantitative PCR, which measures PCR product accumulation through a dual-labeled fluorigenic probe (i.e., TaqMan® probe), and is described in Held et al., Genome Research 6:986-994 (1996).

Differential gene expression can also be identified, or confirmed using the microarray technique. In this method, polynucleotide sequences of interest (including cDNAs and oligonucleotides) are plated, or arrayed, on a microchip substrate. The arrayed sequences are then hybridized with specific DNA probes from cells or tissues of interest. Just as in the RT-PCR method, the source of mRNA typically is total RNA isolated from human tumors or tumor cell lines, such as tumor cell lines with low and high metastatic potential.

The development of microarray methods for large-scale analysis of gene expression makes it possible to search systematically for molecular markers of cancer classification (such as tumors with different metastatic potentials) and outcome prediction in a variety of tumor types.

Alternatively or in addition, differential gene expression between tumors with low and high metastatic potential can be determined or confirmed at the protein level, using proteomics techniques. Proteomics techniques typically include the following steps: (1) separation of individual proteins in a sample by 2-D gel electrophoresis (2-D PAGE); (2) identification of the individual proteins recovered from the gel, e.g. my mass spectrometry or N-terminal sequencing, and (3) analysis of the data using bioinformatics. Proteomics methods are valuable supplements to other methods of gene expression profiling, and can be used, alone or in combination with other methods, to detect the products of the metastasis markers of the present invention.

In the present invention, several tumor markers have been identified by RT-PCR, as described in the Examples, and are listed in Tables 2 and 6 below.

The genes over-expressed or under-expressed in tumor, such as metastatic tumor, are useful diagnostic and prognostic markers of cancer, including metastatic cancer, and targets for therapeutic intervention to treat cancer, including metastatic cancer.

Thus, for example, cancer can be diagnosed by detecting a differentially expressed gene or gene product by methods known in the art, such as, for example, by assays based on nucleic acid hybridization, or by using antibodies to the target gene products in various binding assays, such as competitive binding assays, direct or indirect sandwich assays and immunoprecipitation assays conducted in either heterogeneous or homogeneous phases (Zola, Monoclonal Antibodies: A Manual of Techniques, CRC Press, Inc. (1987) pp. 147-158). The antibodies used in the diagnostic assays can be labeled with a detectable moiety. The detectable moiety should be capable of producing, either directly or indirectly, a detectable signal. For example, the detectable moiety may be a radioisotope, such as ³H, ¹⁴C, ³²P, ³⁵S, or ¹²⁵I, a fluorescent or chemiluminescent compound, such as fluorescein isothiocyanate, rhodamine, or luciferin, or an enzyme, such as alkaline phosphatase, beta-galactosidase or horseradish peroxidase. Any method known in the art for conjugating the antibody to the detectable moiety may be employed, including those methods described by Hunter et al., Nature, 144:945 (1962); David et al., Biochemistry, 13:1014 (1974); Pain et al., J. Immunol. Meth., 40:219 (1981); and Nygren, J. Histochem and Cytochem., 30:407 (1982).

The differentially expressed genes or gene products can also be used for screening drug candidates for the treatment of cancer, including metastatic cancer. The screened drug candidates can be peptides, polypeptides, non-peptide small organic or inorganic molecules, antibodies, including antibody fragments, antisense molecules, oligonucleotide decoy molecules, and the like. In certain embodiments, the screening assays can be performed in a microarray format, which facilitates high throughput screening.

Further details of the invention are illustrated by the following non-limiting examples.

### EXAMPLE 1

Study of Hepatic Metastasis of Human Pancreatic Cancer

Materials and Methods

Male NOG mice and NOD/shiJic-scid mice of 7-9 weeks, which had been obtained from the Central Institute for Experimental Animals (CIEA, Kawasaki, Japan), were used in this study. The animals were kept under specific pathogen-free conditions according to the Guideline for the Regulation of Animal Experimentation of CIEA. All human pancreatic cancer cell lines used in this study were obtained from the American Type Culture Collection (Rockville, Md., USA). Culture media for AsPC-1 and Capan-1 were Dulbecco's modified Eagle's medium (DMEM) supplemented with 20% and 15% fetal bovine serum (FBS, Hyclone), respectively. MIAPaCa-2 and PANC-1 were maintained a culture of DMEM supplemented with 10% FBS. BxPC-3, Capan-2 and PL45 were maintained a culture of RPMI1640 (SIGMA, Cat.No. D6046 or D5796) supplemented with 10% FBS. These were maintained at 37° C. in humidified atmosphere with 5% CO₂. Experimental liver metastases were generated by intrasplenic/portal injection of cancer cells, as described previously (Khatib et al., Cancer Res. 62:242-50 (2002)). The animals were sacrificed 6-8 weeks later and liver metastases were enumerated immediately, without prior fixation. The metastatic lesions were evaluated on the following scale: O=No metastatic lesion; 1=1-10 metastatic lesions; 2=11−20 metastatic lesions; 3=21 or more metastatic lesions.

Results

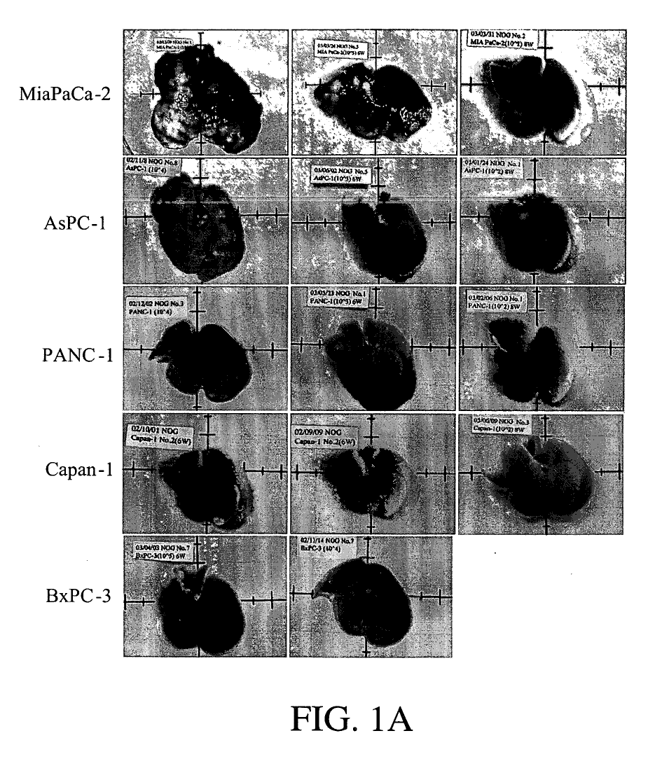

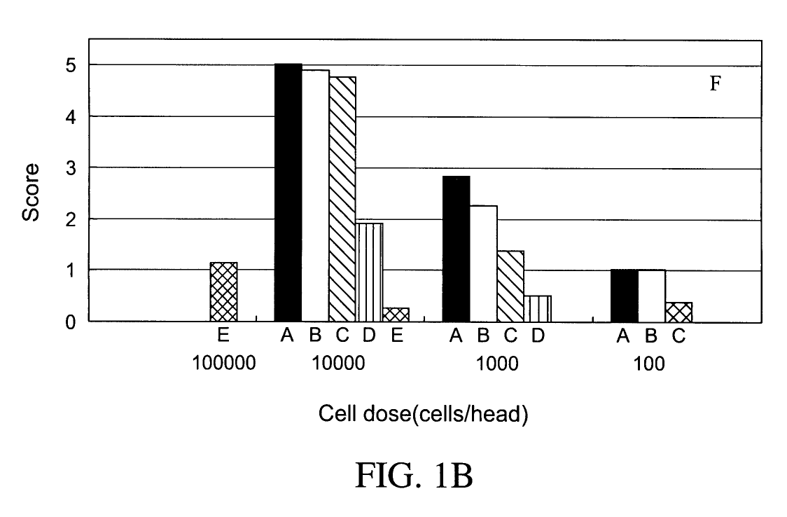

The incidences of hepatic metastases and the number of liver foci in NOG mice were far higher than those in NOD/SCID mice (Table 1 & FIGS. 1A and B). When the mice were inoculated with 1×10⁴ cells and sacrificed 6 weeks later, the incidences of hepatic metastases in NOG mice were as follows:

MIAPaCa-2, AsPC-1 and PANC-1100%;

Capan-1 90%,

BxPC-3 12.5%; and

PL45 and Capan-20%.

In addition, metastases were apparent in 50-80% of NOG mice when 1×10³ MIAPaCa-2, AsPC-1, PANC-1 and Capan-1 cells were inoculated, and even when 1×10² MIAPaCa-2, AsPC-1 and PANC-1 cancer cells were inoculated, 37.5-71.4% of NOG mice show hepatic metastasis. These data indicate that the hepatic metastatic lesions in NOG mice inoculated with human pancreatic cancer cell lines were reproducibly formed in a dose dependent manner.

Typical macroscopic views of liver metastases in NOG mice and in NOD/SCID mice are shown in FIG. 1A. The NOG mice injected with MIAPaCa-2, AsPC-1, PANC-1, Capan-1 and BxPC-3 cells showed multiple round metastases in the liver. However, the numbers of foci in these cell lines were wildly different depending on each cell line. Five out of 7 pancreatic cancer cell lines showed the metastatic potentials in NOG mouse, in contrast, no NOD/SCID mice showed hepatic metastasis under similar conditions, except for AsPC-1. As shown in FIGS. 1A and B, AsPC-1 showed the metastatic potentials in both mice lines, however, the degree of metastases in NOG mice were more severe than those in NOD/SCID mice.

Kusama et al. (Gastroenterology 122:308-17 (2002)) reported that metastatic lesions were apparent in 100% of athymic nude mice injected with 1×10⁶ AsPC-1 cells. These findings suggest AsPC-1 may be one of the cells with high metastatic potential, where the potential is dependent on the cell numbers injected.

The metastatic incidences of NOG mice inoculated with Capan-1 or BxPC-3 were faded away with decreasing the number of inoculating cells. In contrast, metastatic incidences were apparent in more than 50% of NOG mice inoculated with MIAPaCa-2 or AsPC-1 even when NOG mice were inoculated with only 1×10² cells (Table 1). These findings clearly indicate that NOG mice represent a highly superior metastasis model relative to other immunodeficient mouse models, and in particular NOD/SCID mice.

Most previous publications concerning hepatic metastases of human pancreatic cancer cells using nude mice report the intrasplenal inoculation of more than one million cancer cells (Shishido et al., Surg. Today 29(6):519-25 (1999); Nomura et al., Clin. Exp. Metastasis 19:391-9 (2002); and Ikeda et al., Jpn. J. Cancer Res. 81:987-93 (1990)). There are few reports of 100% metastatic incidences, unless high metastatic clones derived from those cells lines were established. However, it is unlikely that more than 1 million cancer cells enter the liver at a stretch via the portal vein and form metastatic foci in pancreatic cancer patients, therefore, the current metastatic animal models are not representative of a typical human clinical situation.

In contrast, NOG mic represent an effective cancer metastasis model, which properly reflects the clinical conditions and behavior of human pancreatic cancer. Accordingly, the well-organized and reproducible hepatic metastases seen in NOG mice are useful in the study of hepatic metastasis of human pancreatic cancer and are expected to become the preferred model for screening and developing new anti-metastasis drugs.

It was reported that the murine NK activity were compensatory very high in immunodeficient animals such as nude, SCID and NOD/SCID mice, and contributed to the low rate of tumor growth and cancer metastasis (Shpitz et al., Anticancer Res. 14(5A): 1927-34 (1994)). In contrast, Ito et al. (Blood 100:3221-8 (2001)) reported that NOG mice have no T, B and NK cells and decrease macrophage functions and dendritic cells functions. It is suggested that in the metastasis model using NOG mice, the metastatic potentials of cancer cells are detected without complex effects upon the immune system of the host, especially NK activity.

Conclusions

The data presented demonstrate that the NOD/SCID/γ_{c}^{null} mouse model has a high potential to engraft xenogenic cells. Using this model for intrasplenic (portal vein) injection of cancer cells, reliable hepatic metastasis behavior of human pancreatic cells was observed. Four out of seven cell lines showed high hepatic metastatic potential (>80% incidence), and three of the cell lines studied showed low metastatic potential (<20% incidence) in NOG mice 6 weeks after transplantation only with 1×10⁴ cells. Moreover, hepatic metastases were apparent in NOG mice even when 1×10² cells of high metastatic cell lines were inoculated. Thus, the metastatic ability of cancer cells was demonstrated with a wide range of inoculated cell number, extending through 3 logarithmic orders of magnitude. The results also show that the NOG mouse model is clearly superior over the NOD/SCID model, which is currently considered the optimal animal model for study of cancer metastasis.

### EXAMPLE 2

Detection of Cancer Metastasis Related Genes in cDNA Microarray

Materials and Methods

Human pancreatic tumor cell lines, MIAPaCa-2, Panc1, Capan2 and PL45 (available from ATCC) were cultured according to the method described in Example 1. Total RNA was extracted from confluent culture of those cells using TRIZOL reagent (GIBCO BRL). Cy-3 labeled cDNA probes were synthesized from 20 μG of total RNA using Atlas human 1K specific primer set (BD), PowerScript labeling kit (BD), and Cy-3 fluorochrome (Amersham). Then, the probe was hybridized to the Atlas Glass Human 1.0 Microarray (BD) according to manufacturer's instructions.

The differentially expressed genes among the pancreatic tumor cell lines were globally searched using the Atlas Glass Human 1.0 Microarray (BD). The Cy-3 labeled signals were detected and obtained and analyzed the corresponding images by a GM418 array scanner (Takara). The data processing was carried out using Imagene Version 5.5 software. In this experiment, we classified human pancreatic tumor cell lines into two groups based on their metastatic potential. MIAPaCa-2 and Panc1 cell lines were classified into a highly metastatic group, while the other cell lines, Capan2 and PL45, were classified into a non-metastatic group. To compare the expression profiles, the average of the signal values from the “highly metastatic group” array was divided by the average of the signal values from the “non-metastatic group” array. The resulting values are referred to as “gene expression levels”, where a 10-fold difference and higher values were considered significant.

Results

Gene expression profiles of each cell line were recorded in an EXCEL file (ArrayData.xcl). The genes that were over-expressed in the highly metastatic cell lines (MIAPaCa-2 and Panc1) relative to the non-metastatic cell lines (Capan2 and PL45), and genes that were under-expressed in the highly metastatic cell lines relative to the non-metastatic cell lines are listed in Table 2. For example, butyrate response factor 1 gene (BRF1) was expressed over 100,000 times more in cancer cells in the highly metastatic group than in cells in the non-metastatic group. In contrast, over 100,000 times over-expression of transducing-beta-2 subunit gene was seen in cells of the non-metastatic group.

As shown in Table 2, the following genes are significantly over-expressed in highly metastatic cells relative to non-metastatic cells: TIS1 1B protein; prostate differentiation factor (PDF); glycoproteins hormone α-subunit; thrombopoietin (THPO); manic fringe homology (MFNG); complement component 5 (C5); jagged homolog 1 (JAG1); interleukin enhancer-binding factor (ILF); PCAF-associated factor 65 alpha; interleukin-12 α-subunit (IL-12-α); nuclear respiratory factor 1 (NRF1); stem cell factor (SCF); transcription factor repressor protein (PRD1-BF1); and small inducible cytokine subfamily A member 1 (SCYA1).

As shown in Table 2, the following genes are significantly under-expressed in highly metastatic cells relative to non-metastatic cells: transducin P2 subunit; X-ray repair complementing defective repair in Chinese hamster cells 1; putative renal organic anion transporter 1; G1/S-specific cyclin E (CCNE); retinoic acid receptor-s (RARG); S-100 calcium-binding protein A1; neutral amino acid transporter A (SATT); dopachrome tautomerase; ets transcription factor (NERF2); calcium-activated potassium channel β-subunit; CD27BP; keratin 10; 6-O-methylguanine-DNA-methyltransferase (MGMT); xeroderma pigmentosum group A complementing protein (XPA); CDC6-related protein; cell division protein kinase 4; nociceptin receptor; cytochrome P450 XXVIIB1; N-myc proto-oncogene; solute carrier family member 1 (SLC2A1); membrane-associated kinase myt1; casper, a FADD- and caspase-related inducer of apoptosis; and C-src proto-oncogene.

The differential expression of the listed and other genes can be used, for example, in drug screening, to test anti-cancer and/or anti-metastatic drug candidates, and for diagnostic and therapeutic purposes, e.g. using gene transfer approaches.

### EXAMPLE 3

Establishment of a Cell Line with High Metastatic Potential

Example 1 describes the establishment of a hepatic metastatic panel using human pancreatic cancer cells xeno-transplanted into NOG mice. Using this panel, the metastatic potentials of several cell lines have been characterized as shown in Table 1. One of the cell lines characterized is BxPC-3. After intrasplenic injection of this cell line into NOG mice only one in eight mice developed hepatic metastasis, i.e. the metastatic potential of this cell line was only 12.5%.

The present Example describes the development of a cell line with high metastatic potential from BxPC-3.

Materials and Methods

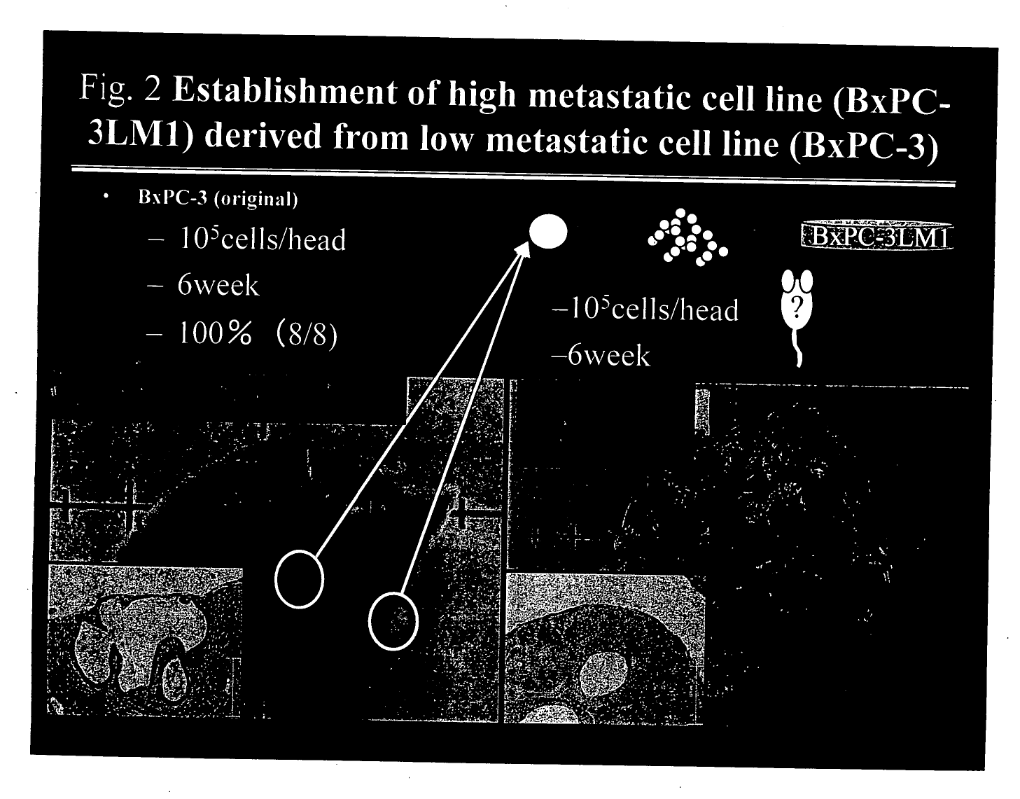

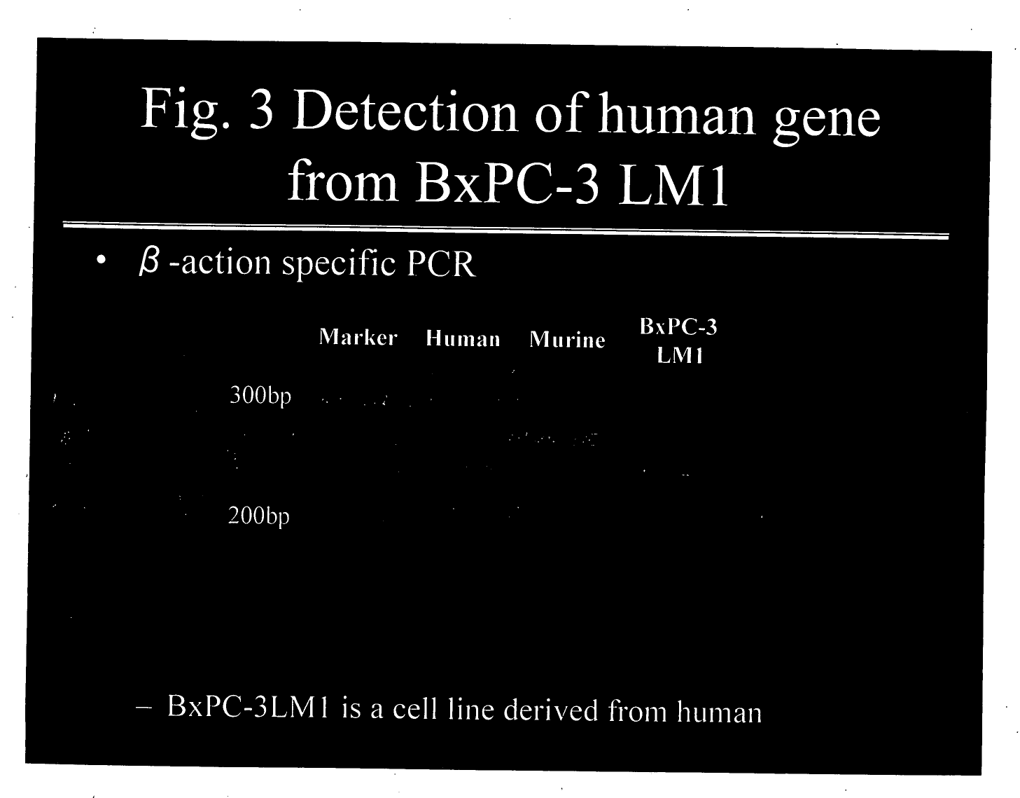

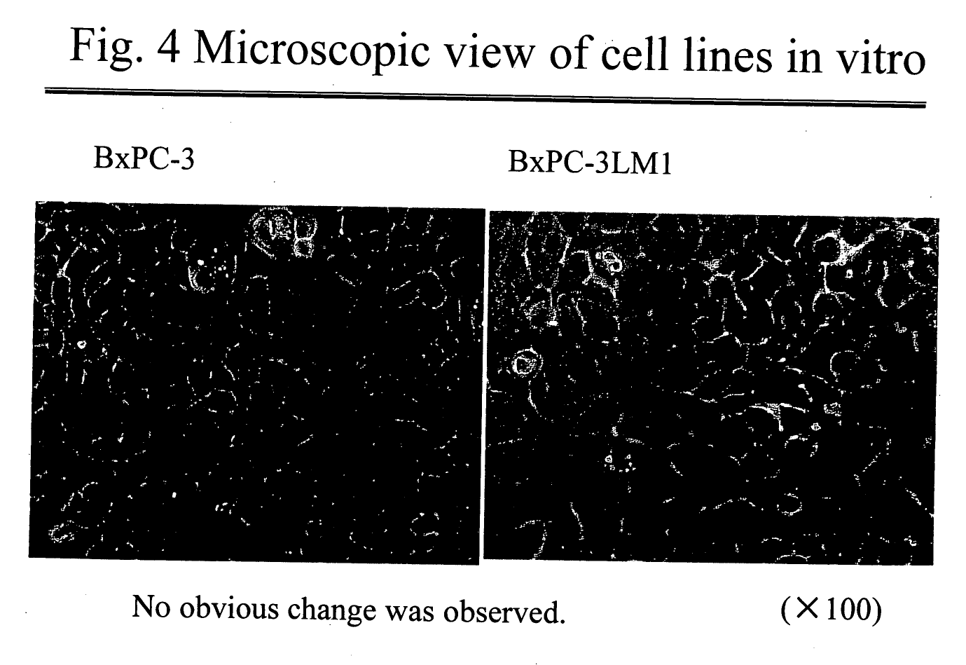

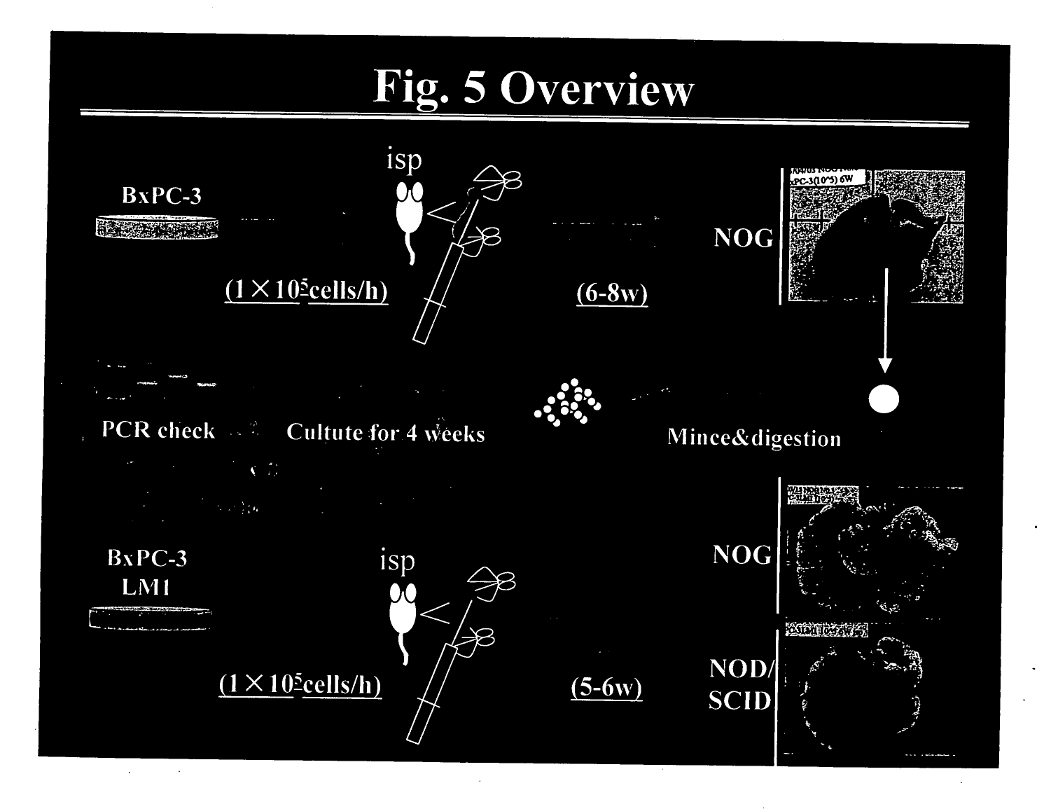

BxPC-3 (1×10⁵ cells) was injected into the spleens of NOG mice. This is barely the amount needed to induce metastasis. After 6-8 weeks, the mice were sacrificed and the livers with a few metastatic foci were harvested. A single cell suspension was prepared by mincing and enzymatic dissociation, and were then cultured in vitro for 4 weeks. The cells in this culture were designated BxPC-3LM1, and the procedure is illustrated in FIGS. 2 and 5.

Results

When the BxPC-3LM1 cell line was analyzed for liver metastatic ability in NOG mice again, high liver metastatic ability was proven (FIG. 2). Table 3 compares the metastatic potentials of the original cell line BxPC-3 and the sub-line BxPC-3LM1 in the liver of the NOG mouse.

In agreement with the data shown in Table 1, 1×10⁴ cells of BxPC-3 resulted in weak metastasis (metastatic score I) in one out of 8 mice, which corresponds to a metastatic incidence of 12.5%. The same dose of BxPC-3ML1 resulted in strong metastasis (scores II and III) in all six mice tested, which translates to a metastatic incidence of 100%.

At a 1×10⁵-cell dose of BxPC-3 resulted in weak liver metastasis in all 8 mice tested (metastatic score: 100%). The same dose of BxPC-3ML1 also yielded a 100% incidence of metastasis, however, with the important difference that all metastases were strong (grade III).

Table 4 compares the metastatic potentials of the original cell line BxPC-3 and the sub-line BxPC-3LM1 in the liver of the NOD/SCID mouse. In this mouse model, 1×10⁴ cells of BxPC-3 resulted in no metastasis in any of the six mice tested, while BxPC-3ML1 resulted in weak metastasis in 3 of the 5 mice tested (60% incidence). The injection of 1×10⁵ cells of BxPC-3 still resulted in no metastasis in the six mice tested in this experiment, while in case of BxPC-3LM1 all mice tested developed strong metastases.

The difference between the metastatic potential of BxPC-3 and BxPC-3LM1 is remarkable, since the latter was obtained by a single transplantation of the BxPC-3 cells in NOG mice.

### EXAMPLE 4

Gene Expression Analysis

As shown by the data set for in Table 5, BxPC-3 and BxPC-3LM1 were similar in their cell number doubling time, in the presence of micro-satellite markers (STR), and ras and p53 mutational status. Accordingly, in its key characteristics, BxPC-3LM1 showed no difference relative to the parental cell line.

The gene expression profiles of BxPC-3 and BxPC-3LM1 were then compared by microarray analysis (GeneChip Human Genome U133 Plus 2.0 Array, Affymetrix), essentially following the procedure described in Example 2.

Results

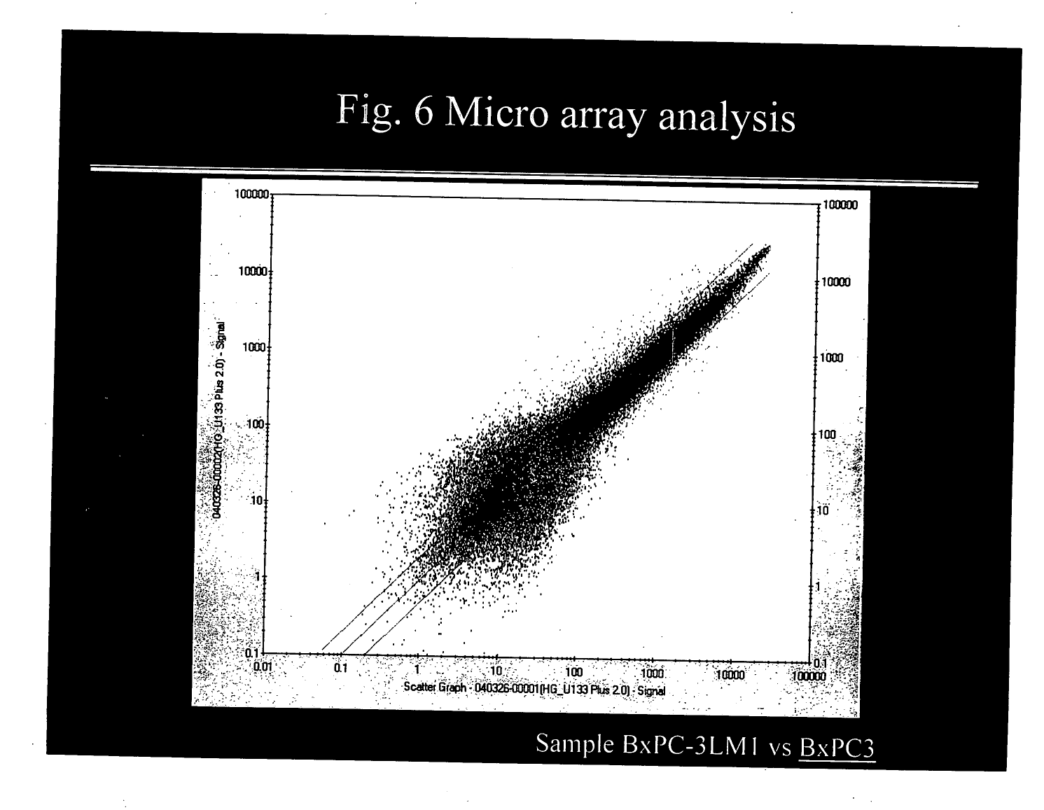

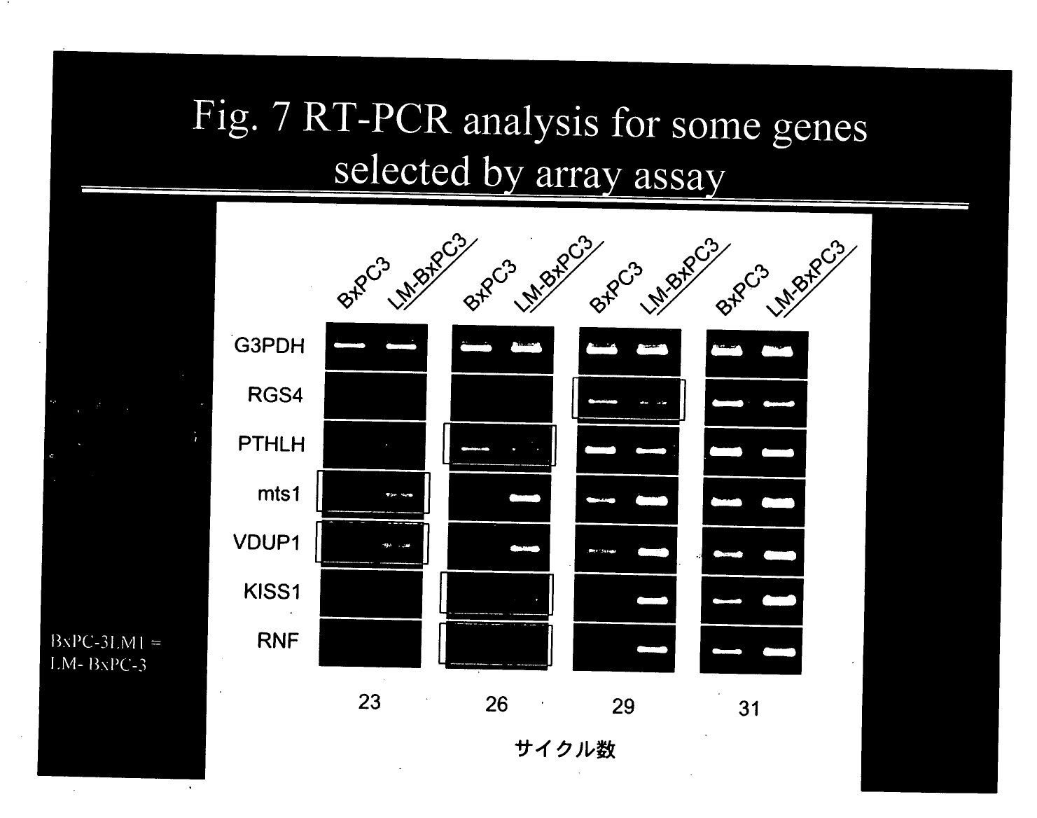

The results of the microarray analysis are shown in FIG. 6, where the signal intensity of each spot of the gene chip microarray is plotted. Table 6 lists all 84 genes selected with this microarray analysis. Some of the genes were further analyzed by RT-PCR. The results of this analysis are shown in FIG. 7.

Discussion

Based on a single transplantation experiment through transplanted in the NOD mice, 84 genes were identified with significantly different gene expressions in the original cell line BxPC-3 and the sub-line BxPC-3LM1 with high metastatic potential. While both cell lines were found to be originated from an identical cell line and to be equivalent pathologically and genetically, and in their in vitro growth. Therefore, the identification of significant differences of the gene expression patterns of the two cell lines are highly significant, and the differentially expressed genes are possibly responsible for metastasis. The differentially expressed genes identified are important targets for the development of drugs and therapeutic approaches for the prevention and treatment of tumor metastasis.

TABLE 1

| | | Cell dose | Autopsy | No. of mice with metastasis/ | Incidence |
|---|---|---|---|---|---|
| Cell line | Mice | (cells/head) | (week) | total no. of mice (h) | (%) |
| MIA PaCa-2 | NOG | 1 × 10⁴ | 6 | 10/10 | 100.0 |
| pancreas; | | 1 × 10³ | 6 | 5/6 | 83.3 |
| adenocarcinoma | | 1 × 10² | 8 | 5/7 | 71.4 |
| | NOD/SCID | 1 × 10⁴ | 6 | 1/10 | 10.0 |
| | | 1 × 10³ | 6 | 0/7 | 0.0 |
| | | 1 × 10² | 8 | 0/6 | 0.0 |
| AsPC-1 | NOG | 1 × 10⁴ | 6 | 9/9 | 100.0 |
| pancreas; metastatic site: | | 1 × 10³ | 6 | 8/8 | 100.0 |
| ascites; adenocarcinoma | | 1 × 10² | 8 | 4/7 | 57.1 |
| | NOD/SCID | 1 × 10⁴ | 6 | 8/9 | 88.9 |
| | | 1 × 10³ | 6 | 1/8 | 12.5 |
| | | 1 × 10² | 8 | 0/6 | 0.0 |
| PANC-1 | NOG | 1 × 10⁴ | 6 | 8/8 | 100.0 |
| pancreas; | | 1 × 10³ | 6 | 6/8 | 75.0 |
| adenocarcinoma | | 1 × 10² | 8 | 3/8 | 37.5 |
| | NOD/SCID | 1 × 10⁴ | 6 | 0/10 | 0.0 |
| | | 1 × 10³ | 6 | 0/6 | 0.0 |
| | | 1 × 10² | 8 | 0/7 | 0.0 |
| Capan-1 | NOG | 1 × 10⁴ | 6 | 9/10 | 90.0 |
| pancreas; metastatic site: | | 1 × 10³ | 6 | 5/10 | 50.0 |
| liver; adenocarcinoma | | 1 × 10² | 8 | 0/8 | 0.0 |
| | NOD/SCID | 1 × 10⁴ | 6 | 0/10 | 0.0 |
| | | 1 × 10³ | 6 | 0/10 | 0.0 |
| | | 1 × 10² | 8 | 0/6 | 0.0 |
| BxPC-3 | NOG | 1 × 10⁵ | 6 | 8/8 | 100.0 |
| pancreas; | | 1 × 10⁴ | 6 | 1/8 | 12.5 |
| adenocarcinoma | NOD/SCID | 1 × 10⁵ | 6 | 0/8 | 0.0 |
| | | 1 × 10⁴ | 6 | 0/6 | 0.0 |
| Capan-2 | NOG | 1 × 10⁵ | 6 | 0/8 | 0.0 |
| pancreas; | | 1 × 10⁴ | 6 | 0/10 | 0.0 |
| adenocarcinoma | NOD/SCID | 1 × 10⁵ | 6 | 0/8 | 0.0 |
| PL45 | NOG | 1 × 10⁵ | 6 | 0/8 | 0.0 |
| Ductal adenocarcinoma; | | 1 × 10⁴ | 6 | 0/10 | 0.0 |
| pancreas | NOD/SCID | 1 × 10⁵ | 6 | 0/8 | 0.0 |

TABLE 2

| Miapaca, | | | | | | | | | | |
|---|---|---|---|---|---|---|---|---|---|---|
| Panc1>>Capan2, | | | | | | | | | | |
| PL45 | Miapaca, Panc1>>Capan2, PL45 | | | | 2High | | | | 2H.AV- | Human |
| MRMCRC | Gene Name | Aspc1 | Miapaca | Panc1 | AVG | Capan2 | PL45 | Low AVG | L.AV | RNA |
| 4176 | TIS11B protein; butyrate response factor | 0.000 | 5.351 | 5.311 | 5.331 | 0.000 | 0.000 | 0.000 | 5.331 | 0.000 |
| | 1 (BRF1); EGF response factor 1 (ERF1) | | | | | | | | | |
| | prostate differentiation factor (PDF); | | | | | | | | | |
| 6272 | macrophage inhibitory cytokine 1 (MIC1); | 0.000 | 3.389 | 3.375 | 3.382 | 0.000 | 0.000 | 0.000 | 3.382 | 0.000 |
| | growth differentiation factor 15 (GDF15) | | | | | | | | | |
| 6274 | glycoprotein hormone alpha subunit | 2.522 | 3.821 | 2.680 | 3.250 | 0.000 | 0.000 | 0.000 | 3.250 | 3.605 |
| | thrombopoietin (THPO); megakaryocyte | | | | | | | | | |
| 6153 | colony stimulating factor; c-mpl ligand; | 2.139 | 3.579 | 2.921 | 3.250 | 0.000 | 0.000 | 0.000 | 3.250 | 3.265 |
| | megakaryocyte growth & development | | | | | | | | | |
| | factor (MGDF) | | | | | | | | | |
| 6322 | manic fringe homolog (MFNG) | 2.512 | 3.578 | 2.757 | 3.167 | 0.000 | 0.000 | 0.000 | 3.167 | 3.021 |
| 6267 | complement component 5 (C5) | 2.158 | 3.911 | 2.373 | 3.142 | 0.000 | 0.000 | 0.000 | 3.142 | 2.893 |
| 6371 | jagged homolog 1 (JAG1; hJ1) | 0.000 | 3.913 | 2.356 | 3.135 | 0.000 | 0.000 | 0.000 | 3.135 | 1.980 |
| | interleukin enhancer-binding factor (ILF) | | | | | | | | | |
| 4262 | ILF + interleukin enhancer binding factor | 2.788 | 3.209 | 3.029 | 3.119 | 0.000 | 0.000 | 0.000 | 3.119 | 0.000 |
| | 2 (ILF2) + interleukin enhancer binding | | | | | | | | | |
| | factor 3 (ILF3) | | | | | | | | | |
| 4261 | PCAF-associated factor 65 alpha | 2.168 | 3.193 | 2.989 | 3.091 | 0.000 | 0.000 | 0.000 | 3.091 | 2.812 |
| | interleukin 12 alpha subunit (IL12-alpha; | | | | | | | | | |
| 6227 | IL12A); cytotoxic lymphocyte maturation | 0.000 | 3.376 | 2.779 | 3.078 | 0.000 | 0.000 | 0.000 | 3.078 | 0.000 |
| | factor 35-kDa subunit (CLMF p35); NK | | | | | | | | | |
| | cell stimulatory factor subunit 1 (NKSF1) | | | | | | | | | |
| 4257 | nuclear respiratory factor 1 (NRF1); alpha | 1.568 | 2.893 | 3.178 | 3.035 | 0.000 | 0.000 | 0.000 | 3.035 | 2.606 |
| | palindromic binding protein | | | | | | | | | |
| 6154 | stem cell factor (SCF); mast cell growth | 0.000 | 3.497 | 2.558 | 3.027 | 0.000 | 0.000 | 0.000 | 3.027 | 2.394 |
| | factor (MGF); c-kit ligand | | | | | | | | | |
| | transcription repressor protein PRDI-BF1; | | | | | | | | | |
| 4251 | beta-interferon gene positive regulatory | 0.000 | 3.233 | 2.812 | 3.023 | 0.000 | 0.000 | 0.000 | 3.023 | 2.473 |
| | domain I binding factor; BLIMP1 | | | | | | | | | |
| | small inducible cytokine subfamily A | | | | | | | | | |
| 6144 | member 1 (SCYA1); T-cell-secreted | 3.069 | 3.341 | 2.691 | 3.016 | 0.000 | 0.000 | 0.000 | 3.016 | 0.000 |
| | protein I-309 | | | | | | | | | |
| 2327 | Transducin beta-2 subunit; GTP-binding | 5.315 | 0.000 | 0.000 | 0.000 | 5.307 | 5.170 | 5.239 | −5.239 | 4.991 |
| | protein G(i)/G(s)/G(t) beta subunit 2 | | | | | | | | | |
| 3366 | X-ray repair-complementing defective | 3.694 | 0.000 | 0.000 | 0.000 | 4.023 | 3.457 | 3.740 | −3.740 | 4.468 |
| | repair in Chinese hamster cells 1 | | | | | | | | | |
| 1433 | putative renal organic anion transporter 1 | 3.765 | 0.000 | 0.000 | 0.000 | 3.857 | 3.594 | 3.726 | −3.726 | 4.873 |
| | (hROAT1) | | | | | | | | | |
| 1227 | G1/S-specific cyclin E (CCNE) | 3.823 | 0.000 | 0.000 | 0.000 | 3.840 | 3.435 | 3.637 | −3.637 | 4.686 |
| 4355 | retinoic acid receptor gamma (RARG) | 3.104 | 0.000 | 0.000 | 0.000 | 3.843 | 3.400 | 3.621 | −3.621 | 3.678 |
| 2437 | S100 calcium-binding protein A1; S-100 | 3.391 | 0.000 | 0.000 | 0.000 | 3.769 | 3.384 | 3.577 | −3.577 | 4.406 |
| | protein alpha chain | | | | | | | | | |
| 1446 | neutral amino acid transporter A (SATT); | 3.441 | 0.000 | 0.000 | 0.000 | 3.373 | 3.580 | 3.476 | −3.476 | 3.938 |
| | alanine/serine/cysteine/threonine | | | | | | | | | |
| | transporter (ASCT1) | | | | | | | | | |
| 6434 | dopachrome tautomerase; dopachrome | 3.620 | 0.000 | 0.000 | 0.000 | 3.536 | 3.332 | 3.434 | −3.434 | 4.262 |
| | delta-isomerase | | | | | | | | | |
| 4312 | ets transcription factor; NERF2 | 3.480 | 0.000 | 0.000 | 0.000 | 3.546 | 3.301 | 3.424 | −3.424 | 2.477 |
| | calcium-activated potassium channel beta | | | | | | | | | |
| 1443 | subunit; maxi K channel beta subunit; BK | 3.608 | 0.000 | 0.000 | 0.000 | 3.680 | 2.944 | 3.312 | −3.312 | 4.554 |
| | channel beta subunit: SLO-beta; | | | | | | | | | |
| | K(VCA)beta | | | | | | | | | |
| 3232 | CD27BP (Siva) | 3.288 | 0.000 | 0.000 | 0.000 | 3.365 | 3.182 | 3.274 | −3.274 | 4.018 |
| 4442 | keratin 10 (KRT10; K10) | 2.521 | 0.000 | 0.000 | 0.000 | 3.505 | 3.010 | 3.257 | −3.257 | 2.721 |
| | 6-O-methylguanine-DNA | | | | | | | | | |
| 3374 | methyltransferase (MGMT); methylated- | 2.848 | 0.000 | 0.000 | 0.000 | 3.030 | 3.475 | 3.252 | −3.252 | 3.275 |
| | DNA-protein-cysteine methyltransferase | | | | | | | | | |
| 3375 | xeroderma pigmentosum group A | 4.009 | 0.000 | 0.000 | 0.000 | 4.284 | 2.200 | 3.242 | −3.242 | 3.592 |
| | complementing protein (XPA) | | | | | | | | | |
| 1347 | CDC6-related protein | 3.995 | 0.000 | 0.000 | 0.000 | 3.390 | 3.040 | 3.215 | −3.215 | 3.093 |
| 1312 | cell division protein kinase 4; cyclin- | 2.522 | 0.000 | 0.000 | 0.000 | 3.201 | 3.205 | 3.203 | −3.203 | 3.332 |
| | dependent kinase 4 (CDK4); PSK-J3 | | | | | | | | | |
| 3432 | nociceptin receptor; orphanin FQ | 3.143 | 0.000 | 0.000 | 0.000 | 3.432 | 2.967 | 3.200 | −3.200 | 4.165 |
| | receptor; opioid receptor kappa 3 | | | | | | | | | |
| | cytochrome P450 XXVIIB1 (CYP27B1); | | | | | | | | | |
| 5456 | 25-hydroxyvitamin D-1-alpha-hydroxylase | 3.603 | 0.000 | 0.000 | 0.000 | 3.346 | 3.049 | 3.197 | −3.197 | 3.856 |
| | (VDR); pseudo-vitamin D dependency | | | | | | | | | |
| | rickets protein 1 (VDDR I) | | | | | | | | | |
| 1174 | N-myc proto-oncogene | 2.414 | 0.000 | 0.000 | 0.000 | 3.323 | 3.012 | 3.167 | −3.167 | 3.466 |
| 1442 | solute carrier family 2 member 1 | 2.961 | 0.000 | 0.000 | 0.000 | 3.215 | 3.059 | 3.137 | −3.137 | 4.077 |
| | (SLC2A1); glucose transporter 1 (GLUT1) | | | | | | | | | |
| 1324 | membrane-associated kinase myt1 | 3.830 | 0.000 | 0.000 | 0.000 | 3.869 | 2.379 | 3.124 | −3.124 | 3.645 |
| | casper, a FADD- and caspase-related | | | | | | | | | |
| 3247 | inducer of apoptosis (CASH-alpha + | 3.352 | 0.000 | 0.000 | 0.000 | 3.531 | 2.659 | 3.095 | −3.095 | 4.255 |
| | CASH-beta); FLAME-1; FLICE-like | | | | | | | | | |
| 1241 | C-src proto-oncogene (SRC1) | 0.000 | 0.000 | 0.000 | 0.000 | 3.316 | 2.829 | 3.072 | −3.072 | 2.795 |

TABLE 3
Metastatic potentials of BxPC-3 and
BxPC-3LM1 in liver of NOG mouse

| | | Metastatic | No. of | Metastatic score | | | |
|---|---|---|---|---|---|---|---|
| Cell Dose | | incidence | mice | (head) | | | |
| (cells/head) | Cell line | (%) | (head) | 0 | I | II | III |
| 1 × 10⁴ | BxPC-3 | 12.5 | 8 | 7 | 1 | 0 | 0 |
| | BxPC-3LM1 | 100.0 | 6 | 0 | 0 | 1 | 5 |
| 1 × 10⁵ | BxPC-3 | 100.0 | 8 | 0 | 8 | 0 | 0 |
| | BxPC-3LM1 | 100.0 | 6 | 0 | 0 | 0 | 6 |

TABLE 4
Metastatic potentials of BxPC-3 and
BxPC-3LM1 in liver of NOD/SCID mouse

| | | Metastatic | No. of | Metastatic score | | | |
|---|---|---|---|---|---|---|---|
| Cell Dose | | incidence | mice | (head) | | | |
| (cells/head) | Cell line | (%) | (head) | 0 | I | II | III |
| 1 × 10⁴ | BxPC-3 | 0 | 6 | 6 | 0 | 0 | 0 |
| | BxPC-3LM1 | 60 | 5 | 2 | 3 | 0 | 0 |
| 1 × 10⁵ | BxPC-3 | 0 | 6 | 6 | 0 | 0 | 0 |
| | BxPC-3LM1 | 100 | 6 | 0 | 0 | 1 | 5 |

TABLE 5
Checking of ras, p53 and other gene profiling by PCR

| | | BxPC3 (Parent) | LM-BxPC3 |
|---|---|---|---|
| Doubling time (hrs) | | 38.6 | 38.8 |
| STR markers | D5S818 | 143/143 | 143/143 |
| (size in bp) | D7S820 | 210/222 | 210/222 |
| | D13S317 | 182/182 | 182/182 |
| | D16S539 | 146/155 | 146/155 |
| ras gene mutation | Ha-ras | CAT to CAC | CAT to CAC |
| | | (codon 27) | (codon 27) |
| | K-ras | none | none |
| | N-ras | none | none |
| p53 gene mutation | | TAT (Y) to | TAT (Y) to |
| | | TGT (C) | TGT (C) |
| | | (codon 20) | (codon 20) |

BxPC-3LM1 = LM-BxPC-3

TABLE 6
Genes selected with array analysis

| Probe Set Name | Signal Log Ratio | description |
|---|---|---|
| 207526_s_at | −2.2 | interleukin 1 receptor-like 1 |
| 211756_at | −2.3 | parathyroid hormone-like hormone |
| 1556773_at | −2.3 | parathyroid hormone-like peptide |
| 204337_at | −2.5 | regulator of G-protein signalling 4 |
| 206300_s_at | −2.3 | parathyroid hormone-like hormone |
| 231771_at | −2.1 | gap junction protein, beta 6 |
| 204339_s_at | −2.3 | regulator of G-protein signalling 4 |
| 204338_s_at | −2.9 | regulator of G-protein signalling 4 |
| 206343_s_at | −2.3 | neuregulin 1 isoform SMDF |
| 223779_at | −2.0 | Unknown |
| 211423_s_at | −2.1 | fungal sterol-C5-desaturase homolog |
| 212977_at | −2.0 | G protein-coupled receptor |
| 222486_s_at | −2.2 | METH1 protein (ADAMTS1) |
| 229004_at | −2.7 | METH1 protein (near ADAMTS15) |
| 206237_s_at | −2.3 | neuregulin 1 isoform HRG-beta2 |
| 235392_at | −2.2 | |
| 205535_s_at | −2.1 | BH-protocadherin (brain-heart) |
| 201008_s_at | 2.4 | upregulated by 1,25-dihydroxyvitamin D-3 |
| 212531_at | 2.4 | lipocalin 2 (oncogene 24p3) (LCN2) |
| 201010_s_at | 2.3 | upregulated by 1,25-dihydroxyvitamin D-3 |
| 201009_s_at | 2.7 | upregulated by 1,25-dihydroxyvitamin D-3 |
| 207076_s_at | 2.8 | argininosuccinate synthetase (ASS) |
| 209365_s_at | 2.2 | extracellular matrix protein 1 (ECM1) |
| 203186_s_at | 3.3 | S100 calcium-binding protein A4 (S100A4) |
| 219795_at | 2.2 | solute carrier family 6 (neurotransmitter transporter), memb [symbol] |
| 206421_s_at | 2.4 | serine (or cysteine) proteinase Inhibitor, clade B (ovalbumin [symbol] |
| 201860_s_at | 2.5 | plasminogen activator, tissue (PLAT) |
| 226071_at | 2.4 | Unknown (thrombospondin repeat containing 1 (TSRC1)) |
| 225987_at | 2.4 | EST (DKFZp666M1410) |
| 202917_s_at | 2.9 | S100 calcium-binding protein A8 (calgranulin A) (S100A8) |
| 223179_at | 2.8 | EST (MGC: 10500) |
| 219014_at | 2.0 | placenta-specific 8 (PLAC8) |
| 202748_at | 2.1 | guanylate binding protein 2, interferon-inducible |
| 204259_at | 2.8 | matrix metalloproteinase 7 |
| 213693_s_at | 2.1 | mucin 1, transmembrane |
| 226755_at | 2.5 | EST (nasopharyngeal carcinoma-associated antigen/LOC9 [symbol] |
| 204885_s_at | 2.0 | megakaryocyte potentiating factor precursor |
| 225283_at | 2.6 | arrestin domain containing 4 |
| 210029_at | 2.1 | interferon-gamma-inducible indoleamine 2,3-dioxygenase ( [symbol] |
| 242649_x_at | 2.6 | |
| 242907_at | 2.0 | |
| 218963_s_at | 2.4 | DKFZP434G032 (Keretin 23) |
| 202357_s_at | 3.8 | B-factor, properdin (complement) |
| 219529_at | 2.4 | chloride intracellular channel 3 |
| 206224_at | 2.0 | cystatin SN |
| 206199_at | 3.1 | carcinoembryonic antigen-related cell adhesionmolecule 7 |
| 235515_at | 2.3 | |
| 226622_at | 2.7 | KIAA1359 (mucin 20) |
| 220196_at | 3.4 | hypothetical protein (mucin 16 = CA125) |
| 208170_s_at | 2.3 | ring finger protein |
| 205640_at | 2.0 | aldehyde dehydrogenase 3 family, member B1 |
| 209183_s_at | 2.7 | DKFZp564P1263 |
| 208607_s_at | 2.4 | serum amyloid A2 |
| 213172_at | 2.0 | |
| 205563_at | 2.4 | KISS-1 metastasis-suppressor KISS1), mRNA. (Nuclear) |
| 214456_x_at | 3.2 | serum amyloid A2-alpha |
| 202161_at | 2.1 | protein kinase C-like 1 |
| 232105_at | 2.2 | |
| 226960_at | 2.1 | |
| 219508_at | 3.8 | glycosaminyl (N-acetyl) transferase 3, mucintype |
| 1555349_s_at | 2.0 | integrin like protein, beta 2 (antigen CD18 (p95), lymphocyt [symbol] |
| 229659_s_at | 2.4 | |
| 206125_s_at | 2.1 | kallikrein 8 |
| 229927_at | 3.2 | EST (similar to lamina associated polypeptide 2, in nuclear [symbol] |
| 35148_at | 2.0 | |
| 226158_at | 2.3 | |
| 205597_at | 3.5 | NG22 protein |
| 1555203_s_at | 2.9 | NG22 (CTL4/NEU1 fusion protein) mRNA (slaildosis) |
| 219722_s_at | 2.3 | |
| 231941_s_at | 2.8 | KIAA1359 (Mucin 20) |
| 211906_s_at | 2.8 | SCCA2b (Squamous cell carcinoma antigen (SCCA) SER [symbol] |
| 211848_s_at | 3.1 | carcinoembryonic antigen 2b |
| 226580_at | 2.5 | sphingosine-1-phosphate phosphotase 2 |
| 227480_at | 2.5 | Susi domain containing2 |
| 219630_at | 2.8 | epithelial protein up-regulated in carcinoma, membrane ass [symbol] |
| 204411_at | 2.3 | KIF21B kinesin family member 21B |
| 206198_s_at | 3.2 | carcinoembryonic antigen |
| 1553589_s_at | 2.1 | epithelial protein up-regulated in carcinoma, membrane ass [symbol] |
| 226147_s_at | 3.2 | polymeric immunoglobulin receptor/hepatocellular carcinom [symbol] |
| 205044_at | 2.9 | gamma-aminobutyric acid (GABA) A receptor, pi |
| 205691_at | 3.3 | synaptogyrin 3 |
| 220390_at | 4.9 | NM_024783.1 |
| 202376_at | [symbol] | alpha-1-antichymotrypsin, precursor |
| 205319_at | 4.0 | prostate stem cell antigen |

What is claimed is:

1. A method for predicting the likelihood of tumor metastasis in a patient, comprising determining the expression level of the RNA transcript of one or more genes selected from the group consisting of TIS1 1B protein; prostate differentiation factor (PDF);
glycoproteins hormone α-subunit; thrombopoietin (THPO); manic fringe homology (MFNG); complement component 5 (C5); jagged homolog 1 (JAG1); interleukin enhancer-binding factor (ILF); PCAF-associated factor 65 alpha; interleukin-12 α-subunit (IL-12-α); nuclear respiratory factor 1 (NRF1); stem cell factor (SCF); transcription factor repressor protein (PRD1-BF1); small inducible cytokine subfamily A member 1 (SCYA1), transducin P2 subunit; X-ray repair complementing defective repair in Chinese hamster cells 1; putative renal organic anion transporter 1; G1/S-specific cyclin E (CCNE); retinoic acid receptor-γ (RARG); S-100 calcium-binding protein A1; neutral amino acid transporter A (SATT); dopachrome tautomerase; ets transcription factor (NERF2); calcium-activated potassium channel β-subunit; CD27BP; keratin 10; 6-O-methylguanine-DNA-methyltransferase (MGMT); xeroderma pigmentosum group A complementing protein (XPA); CDC6-related protein; cell division protein kinase 4; nociceptin receptor; cytochrome P450 XXVIIB1; N-myc proto-oncogene; solute carrier family member 1 (SLC2A1); membrane-associated kinase myt1; casper, a FADD- and caspase-related inducer of apoptosis; and C-src proto-oncogene, or their expression products, in a test biological sample comprising cancer cells obtained from said patient, relative to a reference biological sample, wherein differential expression is indicative of an increased likelihood of tumor metastasis.

2. The method of claim 1 wherein the patient is a human.

3. The method of claim 2 wherein differential expression is overexpression in said test biological sample, and said gene is selected from the group consisting of TIS1 1B protein; prostate differentiation factor (PDF); glycoproteins hormone α-subunit; thrombopoietin (THPO); manic fringe homology (MFNG); complement component 5 (C5); jagged homolog 1 (JAG1); interleukin enhancer-binding factor (ILF); PCAF-associated factor 65 alpha; interleukin-12 α-subunit (IL-12-α); nuclear respiratory factor 1 (NRF1);
stem cell factor (SCF); transcription factor repressor protein (PRD1-BF1); and small inducible cytokine subfamily A member 1 (SCYA1).

4. The method of claim 2 wherein differential expression is underexpression in said test biological sample, and said gene is selected from the group consisting of transducin β2 subunit; X-ray repair complementing defective repair in Chinese hamster cells 1; putative renal organic anion transporter 1; G1/S-specific cyclin E (CCNE); retinoic acid receptor-γ (RARG); S-100 calcium-binding protein A1; neutral amino acid transporter A (SATT);
dopachrome tautomerase; ets transcription factor (NERF2); calcium-activated potassium channel β-subunit; CD27BP; keratin 10; 6-O-methylguanine-DNA-methyltransferase (MGMT); xeroderma pigmentosum group A complementing protein (XPA); CDC6-related protein; cell division protein kinase 4; nociceptin receptor; cytochrome P450 XXVIIB 1; N-myc proto-oncogene; solute carrier family member 1 (SLC2A1); membrane-associated kinase myt1; casper, a FADD- and caspase-related inducer of apoptosis; and C-src proto-oncogene.

5. A method for predicting the likelihood of tumor metastasis in a patient, comprising determining the expression level of the RNA transcript of one or more genes selected from the group consisting of interleukin 1 receptor-like 1, parathyroid hormone-like hormone, parathyroid hormone-like peptide, regulator of G-protein signaling 4, gap junction protein beta 6, neuregulin 1 isoform SMDF, fungal sterol-C5-desaturase homolog, G protein-coupled receptor, METH protein (ADAMTS1), METH1 protein (near ADAMTS15), BH-protocadherin (brain-heart), upregulated by 1,25-dihydroxyvitamin D-3, lipocalin 2 (oncogene 24p3) (LCN2), argininosuccinate synthetase (ASS), extrecellular matrix protein 1 (ECM1), S100 calcium-binding protein A4 (S100Z4), solute carrier family 6 (neurotransmitter transporter) mem1, serine (or cysteine) proteinase inhibitor, lade B (ovalbumin), tissue plasminogen activator (PLAT), EST DKFZp666M1410, C100 calcium-binding protein A8 (calgranulin A) (S100A8), EST (MGC:10500), placenta-specific 8 (PLAC8), interferon-inducible guanylate binding protein 2, matrix metalloproteinase 7, mucin 1 (transmembrane), EST nasopharyngal carcinoma-associated antigen/LOC5, megakaryocyte potentiating factor precursor, arrestin domain containing 4, interferon-gamma-inducible indoleamine 2,3-dioxygenase, DKFZP434G031 (Keratin 23), B-factor (properdin, complement), chloride intracellular channel 3, cystatin SN, carcinoembryonic antigen-related cell adhesion molecule 7, KIAA 1358 (mucin 20), hypothetical protein (mucin 16=CA125), ring finger protein, aldehyde dehydrogenase 3 family member B1, DKFZp564P1263, serum amyloid A2, KiSS-1 metastasis-suppressor (KISS1), serum amyloid A2-alpha, protein kinase C-like 1, glucosaminyl (N-acetyl) transferase 3, mucintype, integrin-like protein beta 2 (antigen CD18, p95), kallikrein 8, NG22 protein, KIAA1359′ (mucin 20), SCA2b (squamous cell carcinoma antigen SCCA, SERPINB4), carcinoembryonic antigen 2b, sphingosine-1-phosphate phosphatase 2, Susi domain containing 2, epithelial protein up-regulated in carcinoma, KIF21B kinesin family member 21B, carcinoembryonic antigen, polymeric immunoglobulin receptor/hepatocelullar carcinoma, gamma-aminobutyric acid (GABA) A receptor p1, synaptogyrin 3, NM_{—}024 783.1, alpha-1-antichymotrypsin precursor, and prostate stem cell antigen, relative to a reference biological sample, wherein differential expression is indicative of an increased likelihood of tumor metastasis.

6. The method of any one or claims 1 to 5, wherein the expression level of the RNA transcript(s) of one or more of said genes is detected.

7. The method of claim 6 wherein the expression level of the RNA transcripts of at least two of said genes is detected.

8. The method of claim 6 wherein the expression level of the RNA transcripts of at least 5 of said genes is detected.

9. The method of claim 6 wherein the expression level of the RNA transcripts of at least 10 of said genes is detected.

10. The method of claim 6 wherein the expression level of the RNA transcripts of all of said genes is detected.

11. The method of any one of claims 1 to 5, wherein the level of the expression product(s) of one or more of said genes is detected.

12. A screening assay, comprising
(a) administering a candidate molecule to a tumor cell expressing one or more gene selected from the group consisting of TIS1 1B protein; prostate differentiation factor (PDF); glycoproteins hormone α-subunit; thrombopoietin (THPO); manic fringe homology (MFNG); complement component 5 (C5); jagged homolog 1 (JAG1); interleukin enhancer-binding factor (ILF); PCAF-associated factor 65 alpha; interleukin-12 α-subunit (IL-12-α); nuclear respiratory factor 1 (NRF1); stem cell factor (SCF); transcription factor repressor protein (PRD1-BF1); small inducible cytokine subfamily A member 1 (SCYA1), transducin P2 subunit; X-ray repair complementing defective repair in Chinese hamster cells 1; putative renal organic anion transporter 1; G1/S-specific cyclin E (CCNE); retinoic acid receptor-γ (RARG); S-100 calcium-binding protein A1; neutral amino acid transporter A (SATT); dopachrome tautomerase; ets transcription factor (NERF2); calcium-activated potassium channel β-subunit; CD27BP; keratin 10; 6-O-methylguanine-DNA-methyltransferase (MGMT); xeroderma pigmentosum group A complementing protein (XPA); CDC6-related protein; cell division protein kinase 4; nociceptin receptor; cytochrome P450 XXVIIB1; N-myc proto-oncogene; solute carrier family member 1 (SLC2A1); membrane-associated kinase myt1; casper, a FADD- and caspase-related inducer of apoptosis; and C-src proto-oncogene, and
(b) determining the effect of said candidate molecule on the expression level of said gene or genes,
wherein said candidate molecule is identified as an anticancer agent, if (i) it is capable of suppressing the expression of one or more gene selected from the group consisting of TIS1 1B protein; prostate differentiation factor (PDF); glycoproteins hormone α-subunit; thrombopoietin (THPO); manic fringe homology (MFNG); complement component 5 (C5); jagged homolog 1 (JAG1); interleukin enhancer-binding factor (ILF); PCAF-associated factor 65 alpha; interleukin-12 α-subunit (IL-12-α); nuclear respiratory factor 1 (NRF1); stem cell factor (SCF); transcription factor repressor protein (PRD1-BF1); and small inducible cytokine subfamily A member 1 (SCYA1), or if (ii) it is capable of increasing the expression of one or more genes selected from the group consisting of transducin β2 subunit; X-ray repair complementing defective repair in Chinese hamster cells 1; putative renal organic anion transporter 1; G1/S-specific cyclin E (CCNE); retinoic acid receptor-γ (RARG); S-100 calcium-binding protein A1; neutral amino acid transporter A (SATT); dopachrome tautomerase; ets transcription factor (NERF2); calcium-activated potassium channel β-subunit; CD27BP; keratin 10; 6-O-methylguanine-DNA-methyltransferase (MGMT); xeroderma pigmentosum group A complementing protein (XPA); CD C6-related protein; cell division protein kinase 4; nociceptin receptor; cytochrome P450 XXVIIB 1; N-myc proto-oncogene; solute carrier family member 1 (SLC2A1); membrane-associated kinase myt1; casper, a FADD- and caspase-related inducer of apoptosis; and C-src proto-oncogene.

13. A screening assay, comprising
(a) administering a candidate molecule to a tumor cell expressing one or more gene selected from the group consisting of interleukin 1 receptor-like 1, parathyroid hormone-like hormone, parathyroid hormone-like peptide, regulator of G-protein signaling 4, gap junction protein beta 6, neuregulin 1 isoform SMDF, fungal sterol-C5-desaturase homolog, G protein-coupled receptor, METH1 protein (ADAMTS1), METH1 protein (near ADAMTS15), BH-protocadherin (brain-heart), upregulated by 1,25-dihydroxyvitamin D-3, lipocalin 2 (oncogene 24p3) (LCN2), argininosuccinate synthetase (ASS), extrecellular matrix protein 1 (ECM1), S100 calcium-binding protein A4 (S100Z4), solute carrier family 6 (neurotransmitter transporter), mem1, serine (or cysteine) proteinase inhibitor, clade B (ovalbumin), tissue plasminogen activator (PLAT), EST DKFZp666M1410, C100 calcium-binding protein A8 (calgranulin A) (S100A8), EST (MGC:10500), placenta-specific 8 (PLAC8), interferon-inducible guanylate binding protein 2, matrix metalloproteinase 7, mucin 1 (transmembrane), EST nasopharyngal carcinoma-associated antigen/LOC5, megakaryocyte potentiating factor precursor, arrestin domain containing 4, interferon-gamma-inducible indoleamine 2,3-dioxygenase, DKFZP434G031 (Keratin 23), B-factor (properdin, complement), chloride intracellular channel 3, cystatin SN, carcinoembryonic antigen-related cell adhesion molecule 7, KIAA1358 (mucin 20), hypothetical protein (mucin 16=CA125), ring finger protein, aldehyde dehydrogenase 3 family member B1, DKFZp564P1263, serum amyloid A2, KiSS-1 metastasis-suppressor (KISS 1), serum amyloid A2-alpha, protein kinase C-like 1, glucosaminyl (N-acetyl) transferase 3, mucintype, integrin-like protein beta 2 (antigen CD18, p95), kallikrein 8, NG22 protein, KIAA1359 (mucin 20), SCA2b (squamous cell carcinoma antigen SCCA, SERPINB4), carcinoembryonic antigen 2b, sphingosine-1-phosphate phosphatase 2, Susi domain containing 2, epithelial protein up-regulated in carcinoma, KIF21B kinesin family member 21B, carcinoembryonic antigen, polymeric immunoglobulin receptor/hepatocelullar carcinoma, gamma-aminobutyric acid (GABA) A receptor p1, synaptogyrin 3, NM_{—}024 783.1, alpha-1-antichymotrypsin precursor, and prostate stem cell antigen, relative to a reference biological sample, wherein differential expression is indicative of an increased likelihood of tumor metastasisinterleukin 1 receptor-like 1, parathyroid hormone-like hormone, parathyroid hormone-like peptide, regulator of G-protein signaling 4, gap junction protein beta 6, neuregulin 1 isoform SMDF, fungal sterol-C5-desaturase homolog, G protein-coupled receptor, METH1 protein (ADAMTS1), METH1 protein (near ADAMTS15), BH-protocadherin (brain-heart), upregulated by 1,25-dihydroxyvitamin D-3, lipocalin 2 (oncogene 24p3) (LCN2), argininosuccinate synthetase (ASS), extrecellular matrix protein 1 (ECM1), S100 calcium-binding protein A4 (S100Z4), solute carrier family 6 (neurotransmitter transporter) mem1, serine (or cysteine) proteinase inhibitor, clade B (ovalbumin), tissue plasminogen activator (PLAT), EST DKFZp666M1410, C100 calcium-binding protein A8 (calgranulin A) (S100A8), EST (MGC:10500), placenta-specific 8 (PLAC8), interferon-inducible guanylate binding protein 2, matrix metalloproteinase 7, mucin 1 (transmembrane), EST nasopharyngal carcinoma-associated antigen/LOC5, megakaryocyte potentiating factor precursor, arrestin domain containing 4, interferon-gamma-inducible indoleamine 2,3-dioxygenase, DKFZP434G031 (Keratin 23), B-factor (properdin, complement), chloride intracellular channel 3, cystatin SN, carcinoembryonic antigen-related cell adhesion molecule 7, KIAA1358 (mucin 20), hypothetical protein (mucin 16=CA125), ring finger protein, aldehyde dehydrogenase 3 family member B1, DKFZp564P1263, serum amyloid A2, KiSS-1 metastasis-suppressor (KISS1), serum amyloid A2-alpha, protein kinase C-like 1, glucosaminyl (N-acetyl) transferase 3, mucintype, integrin-like protein beta 2 (antigen CD18, p95), kallikrein 8, NG22 protein, KIAA1359 (mucin 20), SCA2b (squamous cell carcinoma antigen SCCA, SERPINB4), carcinoembryonic antigen 2b, sphingosine-1-phosphate phosphatase 2, Susi domain containing 2, epithelial protein up-regulated in carcinoma, KIF21B kinesin family member 21B, carcinoembryonic antigen, polymeric immunoglobulin receptor/hepatocelullar carcinoma, gamma-aminobutyric acid (GABA) A receptor p1, synaptogyrin 3, NM_{—}024 783.1, alpha-1-antichymotrypsin precursor, and prostate stem cell antigen, relative to a reference biological sample, wherein differential expression is indicative of an increased likelihood of tumor metastasis, and
(b) determining the effect of said candidate molecule on the expression level of said gene or genes,
wherein said candidate molecule is identified as an anticancer agent, if (i) it is capable of suppressing the expression of one or more gene selected from the group consisting of upregulated by 1,25-dihydroxyvitamin D-3, lipocalin 2 (oncogene 24p3) (LCN2), argininosuccinate synthetase (ASS), extrecellular matrix protein 1 (ECM1), S100 calcium-binding protein A4 (S100Z4), solute carrier family 6 (neurotransmitter transporter) mem1, serine (or cysteine) proteinase inhibitor, clade B (ovalbumin), tissue plasminogen activator (PLAT), EST DKFZp666M1410, C100 calcium-binding protein A8 (calgranulin A) (S100A8), EST (MGC:10500), placenta-specific 8 (PLAC8), interferon-inducible guanylate binding protein 2, matrix metalloproteinase 7, mucin 1 (transmembrane), EST nasopharyngal carcinoma-associated antigen/LOC5, megakaryocyte potentiating factor precursor, arrestin domain containing 4, interferon-gamma-inducible indoleamine 2,3-dioxygenase, DKFZP434G031 (Keratin 23), B-factor (properdin, complement), chloride intracellular channel 3, cystatin SN, carcinoembryonic antigen-related cell adhesion molecule 7, KIAA1358 (mucin 20), hypothetical protein (mucin 16=CA125), ring finger protein, aldehyde dehydrogenase 3 family member B1, DKFZp564P1263, serum amyloid A2, KiSS-1 metastasis-suppressor (KISS1), serum amyloid A2-alpha, protein kinase C-like 1, glucosaminyl (N-acetyl) transferase 3, mucintype, integrin-like protein beta 2 (antigen CD18, p95), kallikrein 8, NG22 protein, KIAA1359 (mucin 20), SCA2b (squamous cell carcinoma antigen SCCA, SERPINB4), carcinoembryonic antigen 2b, sphingosine-1-phosphate phosphatase 2, Susi domain containing 2, epithelial protein up-regulated in carcinoma, KIF21B kinesin family member 21B, carcinoembryonic antigen, polymeric immunoglobulin receptor/hepatocelullar carcinoma, gamma-aminobutyric acid (GABA) A receptor p1, synaptogyrin 3, NM_{—}024 783.1, alpha-1-antichymotrypsin precursor, and prostate stem cell antigen, relative to a reference biological sample, wherein differential expression is indicative of an increased likelihood of tumor metastasisinterleukin 1 receptor-like 1, parathyroid hormone-like hormone, parathyroid hormone-like peptide, regulator of G-protein signaling 4, gap junction protein beta 6, neuregulin 1 isoform SMDF, fungal sterol-C5-desaturase homolog, G protein-coupled receptor, METH1 protein (ADAMTS1), METH1 protein (near ADAMTS15), BH-protocadherin (brain-heart), upregulated by 1,25-dihydroxyvitamin D-3, lipocalin 2 (oncogene 24p3) (LCN2), argininosuccinate synthetase (ASS), extrecellular matrix protein 1 (ECM1), S100 calcium-binding protein A4 (S100Z4), solute carrier family 6 (neurotransmitter transporter) mem1, serine (or cysteine) proteinase inhibitor, clade B (ovalbumin), tissue plasminogen activator (PLAT), EST DKFZp666M1410, C100 calcium-binding protein A8 (calgranulin A) (S100A8), EST (MGC:10500), placenta-specific 8 (PLAC8), interferon-inducible guanylate binding protein 2, matrix metalloproteinase 7, mucin 1 (transmembrane), EST nasopharyngal carcinoma-associated antigen/LOC5, megakaryocyte potentiating factor precursor, arrestin domain containing 4, interferon-gamma-inducible indoleamine 2,3-dioxygenase, DKFZP434G031 (Keratin 23), B-factor (properdin, complement), chloride intracellular channel 3, cystatin SN, carcinoembryonic antigen-related cell adhesion molecule 7, KIAA1358 (mucin 20), hypothetical protein (mucin 16=CA125), ring finger protein, aldehyde dehydrogenase 3 family member B1, DKFZp564P1263, serum amyloid A2, KiSS-1 metastasis-suppressor (KISS1), serum amyloid A2-alpha, protein kinase C-like 1, glucosaminyl (N-acetyl) transferase 3, mucintype, integrin-like protein beta 2 (antigen CD18, p95), kallikrein 8, NG22 protein, KLAA1359 (mucin 20), SCA2b (squamous cell carcinoma antigen SCCA, SERPINB4), carcinoembryonic antigen 2b, sphingosine-1-phosphate phosphatase 2, Susi domain containing 2, epithelial protein up-regulated in carcinoma, KIF21B kinesin family member 21B, carcinoembryonic antigen, polymeric immunoglobulin receptor/hepatocelullar carcinoma, gamma-aminobutyric acid (GABA) A receptor p1, synaptogyrin 3, NM_{—}024 783.1, alpha-1-antichymotrypsin precursor, and prostate stem cell antigen, or if (ii) it is capable of increasing the expression of one or more genes selected from the group consisting of interleukin 1 receptor-like 1, parathyroid hormone-like hormone, parathyroid hormone-like peptide, regulator of G-protein signaling 4, gap junction protein beta 6, neuregulin 1 isoform SMDF, fungal sterol-C5-desaturase homolog, G protein-coupled receptor, METH1 protein (ADAMTS1), METH1 protein (near ADAMTS15), and BH-protocadherin (brain-heart).

14. An array comprising at least one gene, or its expression product, selected from the group consisting of TIS1 1B protein; prostate differentiation factor (PDF); glycoproteins hormone α-subunit; thrombopoietin (THPO); manic fringe homology (MENG); complement component 5 (C5); jagged homolog 1 (JAG1); interleukin enhancer-binding factor (ILF); PCAF-associated factor 65 alpha; interleukin-12 α-subunit (IL-12-α); nuclear respiratory factor 1 (NRF1); stem cell factor (SCF); transcription factor repressor protein (PRDI-BF1); small inducible cytokine subfamily A member 1 (SCYA1), transducin β2 subunit; X-ray repair complementing defective repair in Chinese hamster cells 1; putative renal organic anion transporter 1; G1/S-specific cyclin E (CCNE); retinoic acid receptor-γ (RARG); S-100 calcium-binding protein A1; neutral amino acid transporter A (SATT); dopachrome tautomerase; ets transcription factor (NERF2); calcium-activated potassium channel β-subunit; CD27BP; keratin 10; 6-O-methylguanine-DNA-methyltransferase (MGMT); xeroderma pigmentosum group A complementing protein (XPA); CDC6-related protein; cell division protein kinase 4; nociceptin receptor; cytochrome P450 XXVIIB1; N-myc proto-oncogene; solute carrier family member 1 (SLC2A1); membrane-associated kinase myt1; casper, a FADD- and caspase-related inducer of apoptosis; and C-src proto-oncogene, or their expression products, immobilized on a solid support.

15. The array of claim 14 comprising all of the following genes: TIS1 1B protein; prostate differentiation factor (PDF); glycoproteins hormone α-subunit; thrombopoietin (THPO); manic fringe homology (MFNG); complement component 5 (C5); jagged homolog 1 (JAG1); interleukin enhancer-binding factor (ILF); PCAF-associated factor 65 alpha; interleukin-12 α-subunit (IL-12-oa); nuclear respiratory factor 1 (NRF1); stem cell factor (SCF); transcription factor repressor protein (PRDI-BF1); small inducible cytokine subfamily A member 1 (SCYA1), or their expression products.

16. The array of claim 14 comprising all of the following genes: transducin P2 subunit; X-ray repair complementing defective repair in Chinese hamster cells 1; putative renal organic anion transporter 1; G1/S-specific cyclin E (CCNE); retinoic acid receptor-γ (RARG); S-100 calcium-binding protein A1; neutral amino acid transporter A (SATT); dopachrome tautomerase; ets transcription factor (NERF2); calcium-activated potassium channel β-subunit; CD27BP; keratin 10; 6-O-methylguanine-DNA-methyltransferase (MGMT); xeroderma pigmentosum group A complementing protein (XPA); CDC6-related protein; cell division protein kinase 4; nociceptin receptor; cytochrome P450 XXVIIB1; N-myc proto-oncogene; solute carrier family member 1 (SLC2A1); membrane-associated kinase myt1; casper, a FADD- and caspase-related inducer of apoptosis; and C-src proto-oncogene, or their expression products.

17. An array comprising at least one gene, or its expression product, selected from the group consisting of interleukin 1 receptor-like 1, parathyroid hormone-like hormone, parathyroid hormone-like peptide, regulator of G-protein signaling 4, gap junction protein beta 6, neuregulin 1 isoform SMDF, fungal sterol-C5-desaturase homolog, G protein-coupled receptor, METH1 protein (ADAMTS1), METH1 protein (near ADAMTS15), BH-protocadherin (brain-heart), upregulated by 1,25-dihydroxyvitamin D-3, lipocalin 2 (oncogene 24p3) (LCN2), argininosuccinate synthetase (ASS), extrecellular matrix protein 1 (ECM1), S100 calcium-binding protein A4 (S100Z4), solute carrier family 6 (neurotransmitter transporter) mem1, serine (or cysteine) proteinase inhibitor, clade B (ovalbumin), tissue plasminogen activator (PLAT), EST DKFZp666M1410, C100 calcium-binding protein A8 (calgranulin A) (S100A8), EST (MGC:10500), placenta-specific 8 (PLAC8), interferon-inducible guanylate binding protein 2, matrix metalloproteinase 7, mucin 1 (transmembrane), EST nasopharyngal carcinoma-associated antigen/LOC5, megakaryocyte potentiating factor precursor, arrestin domain containing 4, interferon-gamma-inducible indoleamine 2,3-dioxygenase, DKFZP434G031 (Keratin 23), B-factor (properdin, complement), chloride intracellular channel 3, cystatin SN, carcinoembryonic antigen-related cell adhesion molecule 7, KIAA1358 (mucin 20), hypothetical protein (mucin 16=CA125), ring finger protein, aldehyde dehydrogenase 3 family member B1, DKFZp564P1263, serum amyloid A2, KiSS-1 metastasis-suppressor (KISS 1), serum amyloid A2-alpha, protein kinase C-like 1, glucosaminyl (N-acetyl) transferase 3, mucintype, integrin-like protein beta 2 (antigen CD18, p95), kallikrein 8, NG22 protein, KIAA1359 (mucin 20), SCA2b (squamous cell carcinoma antigen SCCA, SERPINB4), carcinoembryonic antigen 2b, sphingosine-1-phosphate phosphatase 2, Susi domain containing 2, epithelial protein up-regulated in carcinoma, KIF21B kinesin family member 21B, carcinoembryonic antigen, polymeric immunoglobulin receptor/hepatocelullar carcinoma, gamma-aminobutyric acid (GABA) A receptor p1, synaptogyrin 3, NM_{—}024 783.1, alpha-1-antichymotrypsin precursor, and prostate stem cell antigen, relative to a reference biological sample, wherein differential expression is indicative of an increased likelihood of tumor metastasis, or their expression products, immobilized on a solid support.

18. A screening assay, comprising
determining the ability of a candidate molecule to modulate the expression of one or more gene selected from the group consisting of TIS1 1B protein; prostate differentiation factor (PDF); glycoproteins hormone α-subunit; thrombopoietin (THPO); manic fringe homology (MFNG); complement component 5 (C5); jagged homolog 1 (JAG1); interleukin enhancer-binding factor (ILF); PCAF-associated factor 65 alpha; interleukin-12 α-subunit (IL-12-α); nuclear respiratory factor 1 (NRF1); stem cell factor (SCF); transcription factor repressor protein (PRDI-BF1); small inducible cytokine subfamily A member 1 (SCYA1), transducin P2 subunit; X-ray repair complementing defective repair in Chinese hamster cells 1; putative renal organic anion transporter 1; G1/S-specific cyclin E (CCNE); retinoic acid receptor-s (RARG); S-100 calcium-binding protein A1; neutral amino acid transporter A (SATT); dopachrome tautomerase; ets transcription factor (NERF2); calcium-activated potassium channel O-subunit; CD27BP; keratin 10; 6-O-methylguanine-DNA-methyltransferase (MGMT); xeroderma pigmentosum group A complementing protein (XPA); CDC6-related protein; cell division protein kinase 4; nociceptin receptor; cytochrome P450 XXVIIB1; N-myc proto-oncogene; solute carrier family member 1 (SLC2A1); membrane-associated kinase myt1; casper, a FADD- and caspase-related inducer of apoptosis; and C-src proto-oncogene, and identifying said candidate molecule as an anticancer agent when said modulation occurs.

19. The screening assay of claim 18 wherein said modulation is suppression of the expression of one or more genes selected from the group consisting of TIS1 1B protein; prostate differentiation factor (PDF); glycoproteins hormone α-subunit; thrombopoietin (THPO); manic fringe homology (MFNG); complement component 5 (C5); jagged homolog 1 (JAG1); interleukin enhancer-binding factor (ILF); PCAF-associated factor 65 alpha; interleukin-12 α-subunit (IL-12-α); nuclear respiratory factor 1 (NRF1); stem cell factor (SCF); transcription factor repressor protein (PRD1-BF1); and small inducible cytokine subfamily A member 1 (SCYA1).

20. The screening assay of claim 18 wherein said modulation is an increase in the expression level of one or more genes selected from the group consisting of transducin β2 subunit; X-ray repair complementing defective repair in Chinese hamster cells 1; putative renal organic anion transporter 1; G1/S-specific cyclin E (CCNE); retinoic acid receptor-γ (RARG); S-100 calcium-binding protein A1; neutral amino acid transporter A (SATT); dopachrome tautomerase; ets transcription factor (NERF2); calcium-activated potassium channel β-subunit; CD27BP; keratin 10; 6-O-methylguanine-DNA-methyltransferase (MGMT); xeroderma pigmentosum group A complementing protein (XPA); CDC6-related protein; cell division protein kinase 4; nociceptin receptor; cytochrome P450 XXVIIB1; N-myc proto-oncogene; solute carrier family member 1 (SLC2A1); membrane-associated kinase myt1; casper, a FADD- and caspase-related inducer of apoptosis; and C-src proto-oncogene.

21. A screening assay, comprising determining the ability of a candidate molecule to modulate the expression of one or more gene selected from the group consisting of interleukin 1 receptor-like 1, parathyroid hormone-like hormone, parathyroid hormone-like peptide, regulator of G-protein signaling 4, gap junction protein beta 6, neuregulin 1 isoform SMDF, fungal sterol-C5-desaturase homolog, G protein-coupled receptor, METH1 protein (ADAMTS1), METH1 protein (near ADAMTS15), BH-protocadherin (brain-heart), upregulated by 1,25-dihydroxyvitamin D-3, lipocalin 2 (oncogene 24p3) (LCN2), argininosuccinate synthetase (ASS), extrecellular matrix protein 1 (ECM1), S100 calcium-binding protein A4 (S100Z4), solute carrier family 6 (neurotransmitter transporter) mem1, serine (or cysteine) proteinase inhibitor, clade B (ovalbumin), tissue plasminogen activator (PLAT), EST DKFZp666M1410, C100 calcium-binding protein A8 (calgranulin A) (S100A8), EST (MGC:10500), placenta-specific 8 (PLAC8), interferon-inducible guanylate binding protein 2, matrix metalloproteinase 7, mucin 1 (transmembrane), EST nasopharyngal carcinoma-associated antigen/LOC5, megakaryocyte potentiating factor precursor, arrestin domain containing 4, interferon-gamma-inducible indoleamine 2,3-dioxygenase, DKFZP434G031 (Keratin 23), B-factor (properdin, complement), chloride intracellular channel 3, cystatin SN, carcinoembryonic antigen-related cell adhesion molecule 7, KIAA1358 (mucin 20), hypothetical protein (mucin 16=CA125), ring finger protein, aldehyde dehydrogenase 3 family member B 1, DKFZp564P1263, serum amyloid A2, KiSS-1 metastasis-suppressor (KISS1), serum amyloid A2-alpha, protein kinase C-like 1, glucosaninyl (N-acetyl) transferase 3, mucintype, integrin-like protein beta 2 (antigen CD18, p95), kallikrein 8, NG22 protein, KIAA1359 (mucin 20), SCA2b (squamous cell carcinoma antigen SCCA, SERPINB4), carcinoembryonic antigen 2b, sphingosine-1-phosphate phosphatase 2, Susi domain containing 2, epithelial protein up-regulated in carcinoma, KIF21B kinesin family member 21B, carcinoembryonic antigen, polymeric immunoglobulin receptor/hepatocelullar carcinoma, gamma-aminobutyric acid (GABA) A receptor p1, synaptogyrin 3, NM_{—}024 783.1, alpha-1-antichymotrypsin precursor, and prostate stem cell antigen, and identifying said candidate molecule as an anticancer agent when said modulation occurs.

22. The screening assay of claim 21 wherein said modulation is suppression of the expression of one or more genes selected from the group consisting of upregulated by 1,25-dihydroxyvitamin D-3, lipocalin 2 (oncogene 24p3) (LCN2), argininosuccinate synthetase (ASS), extrecellular matrix protein 1 (ECM1), S100 calcium-binding protein A4 (S100Z4), solute carrier family 6 (neurotransmitter transporter) mem1, serine (or cysteine) proteinase inhibitor, clade B (ovalbumin), tissue plasminogen activator (PLAT), EST DKFZp666M1410, C100 calcium-binding protein A8 (calgranulin A) (S100A8), EST (MGC:10500), placenta-specific 8 (PLAC8), interferon-inducible guanylate binding protein 2, matrix metalloproteinase 7, mucin 1 (transmembrane), EST nasopharyngal carcinoma-associated antigen/LOC5, megakaryocyte potentiating factor precursor, arrestin domain containing 4, interferon-gamma-inducible indoleamine 2,3-dioxygenase, DKFZP434G031 (Keratin 23), B-factor (properdin, complement), chloride intracellular channel 3, cystatin SN, carcinoembryonic antigen-related cell adhesion molecule 7, KLAA1358 (mucin 20), hypothetical protein (mucin 16=CA125), ring finger protein, aldehyde dehydrogenase 3 family member B1, DKFZp564P1263, serum amyloid A2, KiSS-1 metastasis-suppressor (KISS 1), serum amyloid A2-alpha, protein kinase C-like 1, glucosaminyl (N-acetyl) transferase 3, mucintype, integrin-like protein beta 2 (antigen CD 18, p95), kallikrein 8, NG22 protein, KIAA1359 (mucin 20), SCA2b (squamous cell carcinoma antigen SCCA, SERPINB4), carcinoembryonic antigen 2b, sphingosine-1-phosphate phosphatase 2, Susi domain containing 2, epithelial protein up-regulated in carcinoma, KIF21B kinesin family member 21B, carcinoembryonic antigen, polymeric immunoglobulin receptor/hepatocelullar carcinoma, gamma-aminobutyric acid (GABA) A receptor p1, synaptogyrin 3, NM_{—}024 783.1, alpha-1-antichymotrypsin precursor, and prostate stem cell antigen, relative to a reference biological sample, wherein differential expression is indicative of an increased likelihood of tumor metastasisinterleukin 1 receptor-like 1, parathyroid hormone-like hormone, parathyroid hormone-like peptide, regulator of G-protein signaling 4, gap junction protein beta 6, neuregulin 1 isoform SMDF, fungal sterol-C5-desaturase homolog, G protein-coupled receptor, METH1 protein (ADAMTS1), METH1 protein (near ADAMTS15), BH-protocadherin (brain-heart), upregulated by 1,25-dihydroxyvitamin D-3, lipocalin 2 (oncogene 24p3) (LCN2), argininosuccinate synthetase (ASS), extrecellular matrix protein 1 (ECM1), S100 calcium-binding protein A4 (S100Z4), solute carrier family 6 (neurotransmitter transporter) mem1, serine (or cysteine) proteinase inhibitor, clade B (ovalbumin), tissue plasminogen activator (PLAT), EST DKFZp666MI410, C100 calcium-binding protein A8 (calgranulin A) (S 100A8), EST (MGC: 10500), placenta-specific 8 (PLAC8), interferon-inducible guanylate binding protein 2, matrix metalloproteinase 7, mucin 1 (transmembrane), EST nasopharyngal carcinoma-associated antigen/LOC5, megakaryocyte potentiating factor precursor, arrestin domain containing 4, interferon-gamma-inducible indoleamine 2,3-dioxygenase, DKFZP434G031 (Keratin 23), B-factor (properdin, complement), chloride intracellular channel 3, cystatin SN, carcinoembryonic antigen-related cell adhesion molecule 7, KIAA 1358 (mucin 20), hypothetical protein (mucin 16 CA125), ring finger protein, aldehyde dehydrogenase 3 family member B1, DKFZp564P1263, serum amyloid A2, KiSS-1 metastasis-suppressor-(KISS1), serum amyloid A2-alpha, protein kinase C-like 1, glucosaminyl (N-acetyl) transferase 3, mucintype, integrin-like protein beta 2 (antigen CD18, p95), kallikrein 8, NG22 protein, KIAA1359 (mucin 20), SCA2b (squamous cell carcinoma antigen SCCA, SERPINB4), carcinoembryonic antigen 2b, sphingosine-1-phosphate phosphatase 2, Susi domain containing 2, epithelial protein up-regulated in carcinoma, KIF21B kinesin family member 21B, carcinoembryonic antigen, polymeric immunoglobulin receptor/hepatocelullar carcinoma, gamma-aminobutyric acid (GABA) A receptor p1, synaptogyrin 3, NM_{—}024 783.1, alpha-1-antichymotrypsin precursor, and prostate stem cell antigen.

23. The screening assay of claim 21 wherein said modulation is increase of the expression level of one or more genes selected from the group consisting of interleukin 1 receptor-like 1, parathyroid hormone-like hormone, parathyroid hormone-like peptide, regulator of G-protein signaling 4, gap junction protein beta 6, neuregulin 1 isoform SMDF, fungal sterol-C5-desaturase homolog, G protein-coupled receptor, METH1 protein (ADAMTS1), METH1 protein (near ADAMTS15), and BH-protocadherin (brain-heart).
